SEQUENCE LISTING (11 sequences)

SEQ ID NO: 1 - protein, 624 aa, Homo sapiens
MQPDPRPSGAGACCRFLPLQSQCPEGAGDAVMYASTECKAEVTPSQHGNRTFSYTLEDHTKQAFGIMNELRLSQQLCDVTLQVKYQDAPAAQFMAHKVVLASSSPVFKAMFTNGLREQGMEVVSIEGIHPKVMERLIEFAYTASISMGEKCVLHVMNGAVMYQIDSVVRACSDFLVQQLDPSNAIGIANFAEQIGCVELHQRAREYIYMHFGEVAKQEEFFNLSHCQLVTLISRDDLNVRCESEVFHACINWVKYDCEQRRFYVQALLRAVRCHSLTPNFLQMQLQKCEILQSDSRCKDYLVKIFEELTLHKPTQVMPCRAPKVGRLIYTAGGYFRQSLSYLEAYNPSDGTWLRLADLQVPRSGLAGCVVGGLLYAVGGRNNSPDGNTDSSALDCYNPMTNQWSPCAPMSVPRNRIGVGVIDGHIYAVGGSHGCIHHNSVERYEPERDEWHLVAPMLTRRIGVGVAVLNRLLYAVGGFDGTNRLNSAECYYPERNEWRMITAMNTIRSGAGVCVLHNCIYAAGGYDGQDQLNSVERYDVETETWTFVAPMKHRRSALGITVHQGRIYVLGGYDGHTFLDSVECYDPDTDTWSEVTRMTSGRSGVGVAVTMEPCRKQIDQQNCTC

SEQ ID NO: 2 - DNA, 1872 bp, Homo sapiens
  source 1..1872: mol_type = other DNA
atgcagccagatcccaggcctagcggggctggggcctgctgccgattcctgcccctgcagtcacagtgccctgagggggcaggggacgcggtgatgtacgcctccactgagtgcaaggcggaggtgacgccctcccagcatggcaaccgcaccttcagctacaccctggaggatcataccaagcaggcctttggcatcatgaacgagctgcggctcagccagcagctgtgtgacgtcacactgcaggtcaagtaccaggatgcaccggccgcccagttcatggcccacaaggtggtgctggcctcatccagccctgtcttcaaggccatgttcaccaacgggctgcgggagcagggcatggaggtggtgtccattgagggtatccaccccaaggtcatggagcgcctcattgaattcgcctacacggcctccatctccatgggcgagaagtgtgtcctccacgtcatgaacggtgctgtcatgtaccagatcgacagcgttgtccgtgcctgcagtgacttcctggtgcagcagctggaccccagcaatgccatcggcatcgccaacttcgctgagcagattggctgtgtggagttgcaccagcgtgcccgggagtacatctacatgcattttggggaggtggccaagcaagaggagttcttcaacctgtcccactgccaactggtgaccctcatcagccgggacgacctgaacgtgcgctgcgagtccgaggtcttccacgcctgcatcaactgggtcaagtacgactgcgaacagcgacggttctacgtccaggcgctgctgcgggccgtgcgctgccactcgttgacgccgaacttcctgcagatgcagctgcagaagtgcgagatcctgcagtccgactcccgctgcaaggactacctggtcaagatcttcgaggagctcaccctgcacaagcccacgcaggtgatgccctgccgggcgcccaaggtgggccgcctgatctacaccgcgggcggctacttccgacagtcgctcagctacctggaggcttacaaccccagtgacggcacctggctccggttggcggacctgcaggtgccgcggagcggcctggccggctgcgtggtgggcgggctgttgtacgccgtgggcggcaggaacaactcgcccgacggcaacaccgactccagcgccctggactgttacaaccccatgaccaatcagtggtcgccctgcgcccccatgagcgtgccccgtaaccgcatcggggtgggggtcatcgatggccacatctatgccgtcggcggctcccacggctgcatccaccacaacagtgtggagaggtatgagccagagcgggatgagtggcacttggtggccccaatgctgacacgaaggatcggggtgggcgtggctgtcctcaatcgtctcctttatgccgtggggggctttgacgggacaaaccgccttaattcagctgagtgttactacccagagaggaacgagtggcgaatgatcacagcaatgaacaccatccgaagcggggcaggcgtctgcgtcctgcacaactgtatctatgctgctgggggctatgatggtcaggaccagctgaacagcgtggagcgctacgatgtggaaacagagacgtggactttcgtagcccccatgaagcaccggcgaagtgccctggggatcactgtccaccaggggagaatctacgtccttggaggctatgatggtcacacgttcctggacagtgtggagtgttacgacccagatacagacacctggagcgaggtgacccgaatgacatcgggccggagtggggtgggcgtggctgtcaccatggagccctgccggaagcagattgaccagcagaactgtacctgt

SEQ ID NO: 3 - protein, 134 aa, Homo sapiens
EVTPSQHGNRTFSYTLEDHTKQAFGIMNELRLSQQLCDVTLQVKYQDAPAAQFMAHKVVLASSSPVFKAMFTNGLREQGMEVVSIEGIHPKVMERLIEFAYTASISMGEKCVLHVMNGAVMYQIDSVVRACSDF

SEQ ID NO: 4 - DNA, 402 bp, Homo sapiens
  source 1..402: mol_type = other DNA
gaggtgacgccctcccagcatggcaaccgcaccttcagctacaccctggaggatcataccaagcaggcctttggcatcatgaacgagctgcggctcagccagcagctgtgtgacgtcacactgcaggtcaagtaccaggatgcaccggccgcccagttcatggcccacaaggtggtgctggcctcatccagccctgtcttcaaggccatgttcaccaacgggctgcgggagcagggcatggaggtggtgtccattgagggtatccaccccaaggtcatggagcgcctcattgaattcgcctacacggcctccatctccatgggcgagaagtgtgtcctccacgtcatgaacggtgctgtcatgtaccagatcgacagcgttgtccgtgcctgcagtgacttc

SEQ ID NO: 5 - protein, 297 aa, Homo sapiens
IYTAGGYFRQSLSYLEAYNPSDGTWLRLADLQVPRSGLAGCVVGGLLYAVGGRNNSPDGNTDSSALDCYNPMTNQWSPCAPMSVPRNRIGVGVIDGHIYAVGGSHGCIHHNSVERYEPERDEWHLVAPMLTRRIGVGVAVLNRLLYAVGGFDGTNRLNSAECYYPERNEWRMITAMNTIRSGAGVCVLHNCIYAAGGYDGQDQLNSVERYDVETETWTFVAPMKHRRSALGITVHQGRIYVLGGYDGHTFLDSVECYDPDTDTWSEVTRMTSGRSGVGVAVTMEPCRKQIDQQNCTC

SEQ ID NO: 6 - DNA, 891 bp, Homo sapiens
  source 1..891: mol_type = other DNA
atctacaccgcgggcggctacttccgacagtcgctcagctacctggaggcttacaaccccagtgacggcacctggctccggttggcggacctgcaggtgccgcggagcggcctggccggctgcgtggtgggcgggctgttgtacgccgtgggcggcaggaacaactcgcccgacggcaacaccgactccagcgccctggactgttacaaccccatgaccaatcagtggtcgccctgcgcccccatgagcgtgccccgtaaccgcatcggggtgggggtcatcgatggccacatctatgccgtcggcggctcccacggctgcatccaccacaacagtgtggagaggtatgagccagagcgggatgagtggcacttggtggccccaatgctgacacgaaggatcggggtgggcgtggctgtcctcaatcgtctcctttatgccgtggggggctttgacgggacaaaccgccttaattcagctgagtgttactacccagagaggaacgagtggcgaatgatcacagcaatgaacaccatccgaagcggggcaggcgtctgcgtcctgcacaactgtatctatgctgctgggggctatgatggtcaggaccagctgaacagcgtggagcgctacgatgtggaaacagagacgtggactttcgtagcccccatgaagcaccggcgaagtgccctggggatcactgtccaccaggggagaatctacgtccttggaggctatgatggtcacacgttcctggacagtgtggagtgttacgacccagatacagacacctggagcgaggtgacccgaatgacatcgggccggagtggggtgggcgtggctgtcaccatggagccctgccggaagcagattgaccagcagaactgtacctgt

SEQ ID NO: 7 - protein, 22 aa, unidentified
GGGSGGGSGGGSGGGSGGGSGG

SEQ ID NO: 8 - DNA, 82 bp, unidentified
  source 1..82: mol_type = other DNA
ttttgtaatacgactcactatagggcggccgggaattcgtcgactggatccggtaccgaggagatctgccgccgcgatcgcc

SEQ ID NO: 9 - DNA, 82 bp, unidentified; identical to SEQ ID NO: 8
  source 1..82: mol_type = other DNA

SEQ ID NO: 10 - DNA, 1800 bp, Cypridina noctiluca
  source 1..1800: mol_type = other DNA
gacactatcccggtgatccatacaaaccgaatccaccatgaagacgttaatccttgccgttgcattggtctactgcgtcactgttaactgtcaggaatgtccctacgtagctgaccctccaaacacagttccaacttcctgtgaagctaaagaaggagaatgtattgattccagctgttccacctgcaccagagacatactatcagatggactgtgtgaaaataaaccaggaaagacatgctgtagaatgtgtcaatacgtaattgaatgcagagtagaagctgcgggatggtttagaacattctatggaaagagattccagttccaggaaccaggtacatacgtgttgggtcaaggaaccaagggaggggactggaaagtgtccattacactggagaacctggatggaaccaaaggagctgtgctgaccaagacaagactggaagtggctggagacatcattgacatcgctcaagcaactgagaaccctatcactgtaaacggtggagccgaccccatcatcgccaacccgtacaccatcggtgaggtcaccattgctgttgttgagttgccaggctacaatatcaccgtcattgaattcttcaaactgatcgtgattgacatcctcggaggaagatctgtaaggatcgccccagacacagcaaacaaagggatgatctctggtctgtgtggagatctaaaaatgatggaggataccgacttctcttcagatcccgagcagctcgctattcagcccaaaatcaaccaagaattcgacggttgtccactctatggaaatcctgaagatatcacatactgcaaaggtcttcttgagccatacaaggatagctgccgcaacaacatcaacttctactactacactctgtcctgcgccttcgcccgctgtatgggtggagatgagagagcctcacatgttgtactggactacagggagacgtgcgctgcccccgaaaccagaggtacctgtgttttgtctggacacacattctacgatacatttgacaaagcaagatatcaattccagggtccctgcaaggagattctcatggctgccgactgttattggaacacttgggatgtgaaggtttcacacagaaatgttgactcttacacagaagtagagaaagtacgaatcaggaaacaatccactgtcgtagaactcattgtggatggaaaacagatcctggttggaggagaagccgtgtccatcccttacagctctcagaacacttccatctactggcaagatggcgacatcctgactacagccatcctacctgaagctctggtggtcaagttcaacttcaaacagctgctcgtagtccatatcagagatccatttgatggcaagacctgcggtatttgtggtaactacaaccaggatttcagtgatgattcgtttgatgctgaaggagcctgtgatctgacccccaacccaccaggatgtaccgaagagcagagacctgaggctgaacgactctgtaatagcctatttgtcggtcagagtgatctcgatcaaaaatgcaacgtatgctacaagcctgaccgtgttgaacggtgtatgtacgagtattgcctgaggggacaacagggattctgtgaccacgcatgggagttcaagaaagaatgctacatcaagcatggagacactctagaagtaccagatgaatgcaaataaaaagtacaaaggaatcaaggaaataacggaagaaaaagaagggaaattatctgtgtatgtctatataacgtgtctagttatgcattcattgaaccaagaga

SEQ ID NO: 11 - DNA, 736 bp, Aequorea victoria
  source 1..736: mol_type = other DNA
aagctttattaaaatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataactgcag

(19) United States
(12) Patent Application Publication
(10) Pub. No.: US 2025/0276036 A1
(43) Pub. Date: Sep. 4, 2025

(54) COMPOSITION FOR THE INHIBITION OF NRF2 AND USES THEREOF IN CANCER THERAPY

(71) Applicant: Sunshine Biopharma Inc., Fort Lauderdale, FL (US)

(72) Inventor: Steve N. Slilaty, Laval (CA)

(21) Appl. No.: 18/857,912

(22) PCT Filed: Apr. 20, 2023
(86) PCT No.: PCT/CA2023/050533

§ 371 Date: Oct. 18, 2024

Related U.S. Application Data
(60) Provisional application No. 63/332,728, filed on Apr. 20, 2022.

(51) Int. Cl. A61K 38/17; A61K 9/1272; A61K 31/136; A61K 31/282; A61K 31/337; A61K 31/4745; A61K 31/519; A61K 31/675; A61K 31/704; A61K 31/7068; A61K 33/243; A61K 48/00; A61P 35/00

(52) U.S. Cl. CPC A61K 38/1709; A61K 9/1272; A61K 31/136; A61K 31/282; A61K 31/337; A61K 31/4745; A61K 31/519; A61K 31/675; A61K 31/704; A61K 31/7068; A61K 33/243; A61K 48/0033; A61P 35/00

(57) ABSTRACT

The present document describes pharmaceutical composition comprising a plurality of lipid nanoparticles comprising a cationic lipid, a neutral lipid, a cholesterol, and a PEG lipid, wherein the plurality of lipid nanoparticles has a mean particle size between 80 nm and 160 nm. The plurality of lipid nanoparticles comprises within their core a synthetic messenger ribonucleic acid (mRNA) comprising: a) a 5′-cap structure; b) a 5′-untranslated region (UTR); c) an open reading frame encoding a Kelch-like ECH-associated protein 1 (KEAP1) protein or a fragment thereof, consisting of nucleotides comprising uracil, cytosine, adenine, guanine, a modified uracil, a modified cytosine, a modified adenine, a modified guanine, or combinations thereof; d) a 3′-UTR; and e) a poly(A) region of at least 100 nucleotides in length. The KEAP1 protein or a fragment thereof is operable to bind to and inhibit release of Nuclear factor erythroid 2-related factor 2 (Nrf2), and presents the Nrf2 for ubiquitination and subsequent degradation.

### CROSS-REFERENCE TO RELATED APPLICATIONS

This application claims priority of U.S. provisional patent application No. 63/332,728 filed on Apr. 20, 2022, the specification of which is hereby incorporated by reference in its entirety.

### BACKGROUND

### (a) Field

The subject matter disclosed generally relates to lipid nanoparticles comprising a Kelch-like ECH-associated protein 1 (KEAP1) mRNA, and more specifically lipid nanoparticles comprising a KEAP1 mRNA which comprises a 5′-cap structure, a 5′-untranslated region (UTR), an open reading frame encoding KEAP1 protein or a fragment thereof, which is operable to bind to and inhibit release of Nuclear factor erythroid 2-related factor 2 (Nrf2), and presents the Nrf2 for ubiquitination and subsequent degradation, a 3′-UTR; and a poly(A) region of at least 100 nucleotides in length.

### (b) Related Prior Art

Transcription factor nuclear factor-erythroid 2-related factor 2 (Nrf2) regulates a battery of genes encoding carcinogen-detoxifying enzymes and antioxidant proteins by binding to the antioxidant response element (ARE) promoter regulatory sequence. Under basal conditions, in which the redox homeostasis is maintained in cells, Nrf2 is sequestered in the cytoplasm by a protein known as Keap1, which targets Nrf2 for ubiquitination and degradation by the proteasome, and thus controls both the subcellular localization and steady-state levels of Nrf2. In response to oxidative stress or chemopreventive compounds, Keap1-mediated ubiquitination of Nrf2 is decreased significantly and the Nrf2 pathway is turned on. Thus, Keap1 is a molecular switch that senses various stimuli and turns the Nrf2 pathway on and off. Administration of Nrf2-inducing agents has been shown to result in decreased carcinogenesis in animal models and altered carcinogen metabolism in humans. Clinical interventions have shown that Nrf2 inducers increase cytoprotective enzyme expression, resulting in modulation of aflatoxin disposition.

Interestingly, Nrf2 and its downstream genes are overexpressed in many cancer cell lines and human cancer tissues, giving cancer cells an advantage for survival and growth. Furthermore, Nrf2 is upregulated in resistant cancer cells and is thought to be responsible for acquired chemoresistance. Therefore, it may be necessary to inhibit the Nrf2 pathway during chemotherapy, and thereby favor the action of concomitantly administered chemotherapeutic agents.

Keap1 contains the BTB domain mediating Keap1 homodimer formation, the ‘intervening region’ (IVR) (amino acids 180-314), and the C-terminal Kelch domain that mediates binding to the Neh2 domain of Nrf2. Human Keap1 contains 27 cysteine residues. Three key cysteine residues (C151, C273, and C288) have been identified. C151 is required for several Nrf2 inducers, such as sulforaphane (SFN) and tert-butylhydroquinone, to manifest their effect. Importantly, residues C273 and C288 at the IVR domain are necessary for Keap1 to repress Nrt2. A single cysteine to serine mutation C273S or C288S render Keap1 unable to repress Nrf2. The transgenic expression of mutant Keap1(C273A) and/or Keap1(C288A) protein in Keap1 null mice failed to reverse constitutive Nrt2 activation, indicating that cysteine residues at positions 273 and 288 are essential for Keap1 to repress Nrf2 activity in vivo. This suggests a critical role of the domains of Keap1 in the regulation of the functional interaction of Keap1 with Nrf2.

Activator and inhibitor compounds of Nrf2 are well-known in the art (Robledinos-Antón, Oxidative Medicine and Cellular Longevity. Volume 2019, Article ID 9372182). Pharmacologic inhibitors of NRf2 are believed to act as tumor cells sensitizer, contributing to increase the efficiency of anticancer therapy. However, the mechanism of inhibition of these compounds is either unknown or not specific, and therefore, Nrf2 inhibitors are still far from being translated from bench to bedside. The fine structure of Nrt2 is not well known, and this hampers a straightforward strategy for the in silico analysis of small molecules that might dock to relevant domains of interaction with MAF proteins, ARE enhancer, etc. Up to now, the identification of Nrf2 inhibitors still has not provided highly selective inhibitors.

Therefore, there is a need for the identification of pharmaceutical compositions to modulate Nrf2 activity and prevent or at least negatively influence its activity in diseases.

### SUMMARY

According to an embodiment, there is provided a pharmaceutical composition comprising:
a plurality of lipid nanoparticles comprising a cationic lipid, a neutral lipid, a cholesterol, and a PEG lipid, wherein the plurality of lipid nanoparticles has a mean particle size between 80 nm and 160 nm; and wherein the plurality of lipid nanoparticles comprises within their core a synthetic messenger ribonucleic acid (mRNA) comprising:
   a) a 5′-cap structure;
   b) a 5′-untranslated region (UTR);
   c) an open reading frame encoding a Kelch-like ECH-associated protein 1 (KEAP1) protein or a fragment thereof, consisting of nucleotides comprising uracil, cytosine, adenine, guanine, a modified uracil, a modified cytosine, a modified adenine, a modified guanine, or combinations thereof;
   d) a 3′-UTR; and
   e) a poly(A) region of at least 100 nucleotides in length,
      wherein the KEAP1 protein or a fragment thereof is operable to bind to and inhibit release of Nuclear factor erythroid 2-related factor 2 (Nrt2), and presents the Nrf2 for ubiquitination and subsequent degradation.

The pharmaceutical cationic lipid may be a biodegradable cationic lipid.

The biodegradable cationic lipid may comprise an ester linkage.

The biodegradable cationic lipid may comprise DLin-DMA with an internal ester, DLin-DMA with a terminal ester, DLin-MC3-DMA with an internal ester, or DLin-MC3-DMA with a terminal ester.

The at least one 5′-cap structure may comprise cap1, ARCA, inosine, N1-methyl-guanosine, 2′-fluoro-guanosine, 7-deaza-guanosine, 8-oxo-guanosine, 2-amino-guanosine, LNA-guanosine, 2-azido-guanosine, or a combination thereof.

The at least one 5′-cap structure may be cap0, cap1, or ARCA.

The 3′-UTR may be an alpha-globin 3′-UTR.

The 5′-UTR may comprise a Kozak sequence.

The plurality of lipid nanoparticles may have a mean polydispersity index (PDI) of between 0.02 and 0.2.

The plurality of lipid nanoparticles may have a mean lipid to polynucleotide, ratio (wt/wt) of between 10 and 20.

The modified uracil may be N1-methyl-pseudouridine, pseudouridine, or a combination thereof.

The modified uracil may be 5-methoxy-uracil.

The modified cytidine may be 5-methyl-cytidine.

The open reading frame encoding KEAP1 protein or a fragment thereof may comprise SEQ ID NO: 1.

The open reading frame encoding KEAP1 protein may comprise the nucleotide sequence of SEQ ID NO: 2.

The open reading frame encoding KEAP1 protein or a fragment thereof may be a fragment which comprises a BTB domain of KEAP1 comprising SEQ ID NO: 3 fused to a Kelch domain of KEAP1 comprising SEQ ID NO: 5, and is operable to bind to and inhibit release of Nuclear factor erythroid 2-related factor 2 (Nrf2), and presents the Nrf2 for ubiquitination and subsequent degradation.

The BTB domain of KEAP1 may be functionally linked to the Kelch domain of KEAP1 via a peptide linker.

The peptide linker may comprise about 3 to about 40 amino acid residues.

The peptide linker may comprise the amino acid sequence (GGGGS)ₙ or (GGGS)ₙ wherein n≥1.

According to another embodiment, there is provided a method for the treatment of a disease associated with KEAP1 and/or Nrf2 comprising:
(a) systematically administering to a patient in need thereof a therapeutically effective amount of a first therapeutic agent comprising the pharmaceutical composition of any one of claims 1 to 20; and
(b) administering to the patient in need thereof a therapeutically effective amount of a second therapeutic agent.

The second therapeutic agent may be distinct from the first therapeutic agent.

The second therapeutic agent comprises doxorubicin, cisplatin, carboplatin, docetaxel, paclitaxel, protein-bound paclitaxel, cabazitaxel gemcitabine, camptothecin, daunorubicin, idarubicin, hydoxyurea, decitabine, azacytidine, cytarabine, 5-Fluorouracil, capecitabine, irinotecan, oxaliplatin, trifluridine and tipiracil, mitoxantrone, vinorelbine, pemetrexed, leucovorin, irinotecan, folfirinox, epirubicin, estramustine, cyclophosphamide, trastuzumab, pertuzumab.

The disease associated with KEAP1 and/or Nrf2 may be a cancer. The cancer may be a bladder cancer, a leukemia, a colon cancer, a kidney cancer, a liver cancer, a lung cancer, a pancreatic cancer, a stomach cancer, a esophageal cancer, a skin cancer, a prostate cancer, and a breast cancer.

According to another embodiment, there is provided a use of a therapeutically effective amount of a first therapeutic agent comprising the pharmaceutical composition of the present invention in combination with a therapeutically effective amount of a second therapeutic agent for the treatment of a disease associated with KEAP1 and/or Nrf2.

According to another embodiment, there is provided a use of a first therapeutic agent comprising the pharmaceutical composition of the present invention in combination with a second therapeutic agent for the treatment of a disease associated with KEAP1 and/or Nrf2, in the manufacture of a medicament for the treatment of a disease associated with KEAP1 and/or Nrt2.

According to another embodiment, there is provided a first therapeutic agent comprising the pharmaceutical composition of the present invention, in combination with a therapeutically effective amount of a second therapeutic agent for use in the treatment of a disease associated with KEAP1 and/or Nrf2.

The second therapeutic agent may be distinct from the first therapeutic agent.

The second therapeutic agent may comprise doxorubicin, cisplatin, carboplatin, docetaxel, paclitaxel, protein-bound paclitaxel, cabazitaxel gemcitabine, camptothecin, daunorubicin, idarubicin, hydoxyurea, decitabine, azacytidine, cytarabine, 5-Fluorouracil, capecitabine, irinotecan, oxaliplatin, trifluridine and tipiracil, mitoxantrone, vinorelbine, pemetrexed, leucovorin, irinotecan, folfirinox, epirubicin, estramustine, cyclophosphamide, trastuzumab, pertuzumab.

The disease associated with KEAP1 and/or Nrf2 may be a cancer. The cancer may be a bladder cancer, a leukemia, a colon cancer, a kidney cancer, a liver cancer, a lung cancer, a pancreatic cancer, a stomach cancer, a esophageal cancer, a skin cancer, a prostate cancer, and a breast cancer.

The following terms are defined below.

At various places in the present specification, substituents of compounds of the present disclosure are disclosed in groups or in ranges. It is specifically intended that the present disclosure include each and every individual subcombination of the members of such groups and ranges. For example, the term “C1-6 alkyl” is specifically intended to individually disclose methyl, ethyl, C3 alkyl, C4 alkyl, C5 alkyl, and C6 alkyl.

The terms “nucleic acid” and “nucleic acid molecules” are intended to in their broadest sense, any compound and/or substance that comprise a polymer of nucleotides. These polymers are often referred to as polynucleotides. Exemplary nucleic acids or polynucleotides of the invention include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

In preferred embodiments, the nucleic acid molecule is a messenger RNA (mRNA). As used herein, the term “messenger RNA” (mRNA) refers to any polynucleotide which encodes a polypeptide of interest and which is capable of being translated to produce the encoded polypeptide of interest in vitro, in vivo, in situ or ex vivo. In preferred embodiments, the mRNA encodes a Kelch-like ECH-associated protein 1 (KEAP1) protein or a fragment thereof, which is operable to bind to and inhibit release of Nuclear factor erythroid 2-related factor 2 (Nrf2), and presents the Nrf2 for ubiquitination and subsequent degradation.

Traditionally, the basic components of an mRNA molecule include at least a coding region, a 5′ untranslated region (UTR), a 3′UTR, a 5′ cap and a poly(A) tail. Building on this wild type modular structure, the present invention expands the scope of functionality of traditional mRNA molecules by providing polynucleotides or primary RNA constructs which maintain a modular organization, but which comprise one or more structural and/or chemical modifications or alterations which impart useful properties to the polynucleotide including, in some embodiments, the lack of a substantial induction of the innate immune response of a cell into which the polynucleotide is introduced. As such, modified mRNA molecules of the present invention are termed “mmRNA.” As used herein, a “structural” feature or modification is one in which two or more linked nucleotides are inserted, deleted, duplicated, inverted or randomized in a polynucleotide, primary construct or mmRNA without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

About: As used herein, the term “about” means+/−10% of the recited value.

Administered in combination: As used herein, the term “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Approximately: As used herein, the term approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Compound: As used herein, the term “compound,” is meant to include all stereoisomers, geometric isomers, tautomers, and isotopes of the structures depicted.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an oligonucleotide or polypeptide or may apply to a portion, region or feature thereof.

Cytostatic: As used herein, “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof.

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivery: As used herein, “delivery” refers to the act or manner of delivering a compound, substance, entity, moiety, cargo or payload.

Delivery Agent: As used herein, “delivery agent” refers to any substance which facilitates, at least in part, the in vivo delivery of a polynucleotide, primary construct or mmRNA to targeted cells.

Detectable label: As used herein, “detectable label” refers to one or more markers, signals, or moieties which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the peptides or proteins disclosed herein. They may be within the amino acids, the peptides, or proteins, or located at the N- or C-termini.

Digest: As used herein, the term “digest” means to break apart into smaller pieces or components. When referring to polypeptides or proteins, digestion results in the production of peptides.

Distal: As used herein, the term “distal” means situated away from the center or away from a point or region of interest.

Dosing regimen: As used herein, a “dosing regimen” is a schedule of administration or physician determined regimen of treatment, prophylaxis, or palliative care.

Encapsulate: As used herein, the term “encapsulate” means to enclose, surround or encase.

Engineered: As used herein, embodiments of the invention are “engineered” when they are designed to have a feature or property, whether structural or chemical, that varies from a starting point, wild type or native molecule.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” includes at least a polynucleotide, synthetic mRNA, primary construct or mmRNA and a delivery agent.

Fragment: A “fragment,” as used herein, refers to a portion. For example, fragments of proteins may comprise polypeptides obtained by digesting full-length protein isolated from cultured cells.

Functional: As used herein, a “functional” biological molecule is a biological molecule in a form in which it exhibits a property and/or activity by which it is characterized.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the invention, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the invention, two protein sequences are considered to be homologous if the proteins are at least about 50%, 60%, 70%, 80%, or 90% identical for at least one stretch of at least about 20 amino acids.

Identity: As used herein, the term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically a reduction in the level of an mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Linker: As used herein, a linker refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker may be of sufficient length as to not interfere with incorporation into a nucleic acid sequence. The linker can be used for any useful purpose, such as to form mmRNA multimers (e.g., through linkage of two or more polynucleotides, primary constructs, or mmRNA molecules) or mmRNA conjugates, as well as to administer a payload, as described herein. Examples of chemical groups that can be incorporated into the linker include, but are not limited to, alkyl, alkenyl, alkynyl, amido, amino, ether, thioether, ester, alkylene, heteroalkylene, aryl, or heterocycyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers and derivatives thereof., Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond can be cleaved for example by the use of tris(2-carboxyethyl)phosphine (TCEP), or other reducing agents, and/or photolysis, as well as an ester bond can be cleaved for example by acidic or basic hydrolysis.

Modified: As used herein “modified” refers to a changed state or structure of a molecule of the invention. Molecules may be modified in many ways including chemically, structurally, and functionally. In one embodiment, the mRNA molecules of the present invention are modified by the introduction of non-natural nucleosides and/or nucleotides, e.g., as it relates to the natural ribonucleotides A, U, G, and C. Noncanonical nucleotides such as the cap structures are not considered “modified” although they differ from the chemical structure of the A, C, G, U ribonucleotides.

Naturally occurring: As used herein, “naturally occurring” means existing in nature without artificial aid.

Non-human vertebrate: As used herein, a “non human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Open reading frame: As used herein, “open reading frame” or “ORF” refers to a sequence which does not contain a stop codon in a given reading frame.

Operably linked: As used herein, the phrase “operably linked” refers to a functional connection between two or more molecules, constructs, transcripts, entities, moieties or the like.

Peptide: As used herein, “peptide” is less than or equal to 50 amino acids long, e.g., about 5, 10, 15, 20, 25, 30, 35, 40, 45, or 50 amino acids long.

Patient: As used herein, “patient” refers to a subject who may seek or be in need of treatment, requires treatment, is receiving treatment, will receive treatment, or a subject who is under care by a trained professional for a particular disease or condition.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Joumal of Pharmaceutical Science, 66, 1-19 (1977), each of which is incorporated herein by reference in its entirety.

Pharmaceutically acceptable solvate: The term “pharmaceutically acceptable solvate,” as used herein, means a compound of the invention wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Protein of interest: As used herein, the terms “proteins of interest” or “desired proteins” include those provided herein and fragments, mutants, variants, and alterations thereof.

Proximal: As used herein, the term “proximal” means situated nearer to the center or to a point or region of interest.

Pseudouridine: As used herein, pseudouridine refers to the C-glycoside isomer of the nucleoside uridine. A “pseudouridine analog” is any modification, variant, isoform or derivative of pseudouridine. For example, pseudouridine analogs include but are not limited to 1-carboxymethyl-pseudouridine, 1-propynyl-pseudouridine, 1-taurinomethyl-pseudouridine, 1-taurinomethyl-4-thio-pseudouridine, 1-methylpseudouridine (m1ψ), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydropseudouridine, 2-thio-dihydropseudouridine, 2-methoxyuridine, 2-methoxy-4-thio-uridine, 4-methoxy-pseudouridine, 4-methoxy-2-thio-pseudouridine, N1-methyl-pseudouridine, 1-methyl-3-(3-amino-3-carboxypropyl)pseudouridine (acp3ψ), and 2′-O-methyl-pseudouridine (ψm).

Purified: As used herein, “purify,” “purified,” “purification” means to make substantially pure or clear from unwanted components, material defilement, admixture or imperfection.

Sample: As used herein, the term “sample” or “biological sample” refers to a subset of its tissues, cells or component parts (e.g., body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single mute/single point of contact, i.e., single administration event.

Similarity: As used herein, the term “similarity” refers to the overall relatedness between polymeric molecules, e.g., between polynucleotide molecules (e.g., DNA molecules and/or RNA molecules) and/or between polypeptide molecules.

Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable.

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Synthetic: The term “synthetic” means produced, prepared, and/or manufactured by the hand of man. Synthesis of polynucleotides or polypeptides or other molecules of the present invention may be chemical or enzymatic.

Targeted Cells: As used herein, “targeted cells' refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24 hr period. It may be administered as a single unit dose.

Transcription factor As used herein, the term “transcription factor” refers to a DNA-binding protein that regulates transcription of DNA into RNA, for example, by activation or repression of transcription. Some transcription factors effect regulation of transcription alone, while others act in concert with other proteins. Some transcription factor can both activate and repress transcription under certain conditions. In general, transcription factors bind a specific target sequence or sequences highly similar to a specific consensus sequence in a regulatory region of a target gene. Transcription factors may regulate transcription of a target gene alone or in a complex with other molecules.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1 illustrates a schematic of a synthetic mRNA or primary construct according to an embodiment of the present invention.

FIG. 2 illustrates the effect of cLUC or KEAP1 mRNA transfection on MCF7 or MCF7/MDR cells, according to an embodiment of the present invention.

FIG. 3 illustrates relative KEAP1 expression levels in MCF7 or MCF7/MDR cells pursuant to KEAP 1 mRNA transfection.

FIG. 4 illustrates the effect of cLUC or KEAP1 mRNA transfection on MCF7 or HMEC cells, according to an embodiment of the present invention.

FIG. 5 illustrates the relative eGFP expression levels in MCF7 or HMEC cells pursuant to eGFP mRNA transfection.

FIG. 6 illustrates the relative KEAP1 expression levels in MCF7 or HMEC cells pursuant to KEAP 1 mRNA transfection.

FIG. 7 illustrates the effect of cLUC or KEAP1 mRNA transfection on SUIT2 or OVCAR3 cells, according to an embodiment of the present invention.

FIG. 8 illustrates the relative KEAP1 expression levels in SUIT2 or OVCAR3 cells pursuant to KEAP 1 mRNA transfection

It will be noted that throughout the appended drawings, like features are identified by like reference numerals.

### DETAILED DESCRIPTION

In embodiments there is disclosed a pharmaceutical composition which comprises a plurality of lipid nanoparticles comprising a cationic lipid, a neutral lipid, a cholesterol, and a PEG lipid, wherein the plurality of lipid nanoparticles has a mean particle size between 80 nm and 160 nm; and wherein the plurality of lipid nanoparticles comprises within their core a synthetic messenger ribonucleic acid (mRNA) comprising:
f) a 5′-cap structure;
g) a 5′-untranslated region (UTR);
h) an open reading frame encoding a Kelch-like ECH-associated protein 1 (KEAP1) protein or a fragment thereof, consisting of nucleotides comprising uracil, cytosine, adenine, guanine, a modified uracil, a modified cytosine, a modified adenine, a modified guanine, or combinations thereof;
i) a 3′-UTR; and
j) a poly(A) region of at least 100 nucleotides in length,
   wherein the KEAP1 protein or a fragment thereof is operable to bind to and inhibit release of Nuclear factor erythroid 2-related factor 2 (Nrf2), and presents the Nrf2 for ubiquitination and subsequent degradation.

The amino acid sequence of KEAP1 protein is illustrated below, with the BTB domain; 3-box; IVR domain; Kelch domain identified. Also shown below is a corresponding KEAP1 protein amino acid sequence overlaid with a corresponding nucleotide sequence.

```
MQPDPRPSGA GACCRFLPLQ SQCPEGAGDA VMYASTECKA EVTPSQHGNR TFSYTLEDHT KQAFGIMNEL
RLSQQLCDVT LQVKYQDAPA AQFMAHKVVL ASSSPVFKAM FTNGLREQGM EVVSIEGIHP KVMERLIEFA
YTASISMGEK CVLHVMNGAV MYQIDSVVRA CSDFLVQQLD PSNAIGIANE AEQIGCVELH QRAREYIYMH
FGEVAKQEEF FNLSHCQLVT LISRDDLNVR CESEVFHACI NWVKYDCEQR RFYVQALLRA VRCHSLTPNE
LQMQLQKCEI LQSDSRCKDY LVKIFEELTL HKPTQVMPCR APKVGRLIYT AGGYFRQSLS YLEAYNPSDG
TWLRLADLQV PRSGLAGCVV GGLLYAVGGR NNSPDGNTDS SALDCYNPMT NQWSPCAPMS VPRNRIGVGV
IDGHIYAVGG SHGCIHHNSV ERYEPERDEW HLVAPMLTRR IGVGVAVLNR LLYAVGGEDG TNRLNSAECY
YPERNEWRMI TAMNTIRSGA GVCVLHNCIY AAGGYDGQDQ LNSVERYDVE TETWTFVAPM KHRRSALGIT
VHQGRIYVLG GYDGHTFLDS VECYDPDTDT WSEVTRMTSG RSGVGVAVTM EPCRKQIDQQ NCTC
KAEP1 sequence: BTB domain; 3-box; IVR domain; Kelch domain.
atg cag cca gat ccc agg cct agc ggg gct ggg gcc tgc tgc cga ttc ctg ccc ctg cag
  M Q P D P R P S G A G A C C R F L P L Q
tca cag tgc cct gag ggg gca ggg gac gcg gtg atg tac gcc tcc act gag tgc aag gcg
  S Q C P E G A G D A V M Y A S T E C K A
gag gtg acg ccc tcc cag cat ggc aac cgc acc ttc agc tac acc ctg gag gat cat acc
  E V T P S Q H G N R T F S Y T L E D H T
aag cag gcc ttt ggc atc atg aac gag ctg cgg ctc agc cag cag ctg tgt gac gtc aca
  K Q A F G I M N E L R L S Q Q L C D V T
ctg cag gtc aag tac cag gat gca ccg gcc gcc cag ttc atg gcc cac aag gtg gtg ctg
  L Q V K Y Q D A P A A Q F M A H K V V L
gcc tca tcc agc cct gtc ttc aag gcc atg ttc acc aac ggg ctg cgg gag cag ggc atg
  A S S S P V F K A M F T N G L R E Q G M
gag gtg gtg tcc att gag ggt atc cac ccc aag gtc atg gag cgc ctc att gaa ttc gcc
  E V V S I E G I H P K V M E R L I E F A
tac acg gcc tcc atc tcc atg ggc gag aag tgt gtc ctc cac gtc atg aac ggt gct gtc
  Y T A S I S M G E K C V L H V M N G A V
atg tac cag atc gac agc gtt gtc cgt gcc tgc agt gac ttc ctg gtg cag cag ctg gac
  M Y Q I D S V V R A C S D F L V Q Q L D
ccc agc aat gcc atc ggc atc gcc aac ttc gct gag cag att ggc tgt gtg gag ttg cac
  P S N A I G I A N F A E Q I G C V E L H
cag cgt gcc cgg gag tac atc tac atg cat ttt ggg gag gtg gcc aag caa gag gag ttc
  Q R A R E Y I Y M H F G E V A K Q E E F
ttc aac ctg tcc cac tgc caa ctg gtg acc ctc atc agc cgg gac gac ctg aac gtg cgc
  F N L S H C Q L V T L I S R D D L N V R
tgc gag tcc gag gtc ttc cac gcc tgc atc aac tgg gtc aag tac gac tgc gaa cag cga
  C E S E V F H A C I N W V K Y D C E Q R
cgg ttc tac gtc cag gcg ctg ctg cgg gcc gtg cgc tgc cac tcg ttg acg ccg aac ttc
  R F Y V Q A L L R A V R C H S L T P N F
ctg cag atg cag ctg cag aag tgc gag atc ctg cag tcc gac tcc cgc tgc aag gac tac
  L Q M Q L Q K C E I L Q S D S R C K D Y
ctg gtc aag atc ttc gag gag ctc acc ctg cac aag ccc acg cag gtg atg ccc tgc cgg
  L V K I F E E L T L H K P T Q V M P C R
gcg ccc aag gtg ggc cgc ctg atc tac acc gcg ggc ggc tac ttc cga cag tcg ctc agc
  A P K V G R L I Y T A G G Y F R Q S L S
tac ctg gag gct tac aac ccc agt gac ggc acc tgg ctc cgg ttg gcg gac ctg cag gtg
  Y L E A Y N P S D G T W L R L A D L Q V
ccg cgg agc ggc ctg gcc ggc tgc gtg gtg ggc ggg ctg ttg tac gcc gtg ggc ggc agg
  P R S G L A G C V V G G L L Y A V G G R
aac aac tcg ccc gac ggc aac acc gac tcc agc gcc ctg gac tgt tac aac ccc atg acc
  N N S P D G N T D S S A L D C Y N P M T
aat cag tgg tcg ccc tgc gcc ccc atg agc gtg ccc cgt aac cgc atc ggg gtg ggg gtc
  N Q W S P C A P M S V P R N R I G V G V
atc gat ggc cac atc tat gcc gtc ggc ggc tcc cac ggc tgc atc cac cac aac agt gtg
  I D G H I Y A V G G S H G C I H H N S V
gag agg tat gag cca gag cgg gat gag tgg cac ttg gtg gcc cca atg ctg aca cga agg
  E R Y E P E R D E W H L V A P M L T R R
atc ggg gtg ggc gtg gct gtc ctc aat cgt ctc ctt tat gcc gtg ggg ggc ttt gac ggg
  I G V G V A V L N R L L Y A V G G F D G
aca aac cgc ctt aat tca gct gag tgt tac tac cca gag agg aac gag tgg cga atg atc
  T N R L N S A E C Y Y P E R N E W R M I
aca gca atg aac acc atc cga agc ggg gca ggc gtc tgc gtc ctg cac aac tgt atc tat
  T A M N T I R S G A G V C V L H N C I Y
gct gct ggg ggc tat gat ggt cag gac cag ctg aac agc gtg gag cgc tac gat gtg gaa
  A A G G Y D G Q D Q L N S V E R Y D V E
aca gag acg tgg act ttc gta gcc ccc atg aag cac cgg cga agt gcc ctg ggg atc act
  T E T W T F V A P M K H R R S A L G I T
gtc cac cag ggg aga atc tac gtc ctt gga ggc tat gat ggt cac acg ttc ctg gac agt
  V H Q G R I Y V L G G Y D G H T F L D S
gtg gag tgt tac gac cca gat aca gac acc tgg agc gag gtg acc cga atg aca tcg ggc
  V E C Y D P D T D T W S E V T R M T S G
cgg agt ggg gtg ggc gtg gct gtc acc atg gag ccc tgc cgg aag cag att gac cag cag
  R S G V G V A V T M E P C R K Q I D Q Q
aac tgt acc tgt
N C T C
KAEP1 combined nucleotide and amino acid sequence: BTB domain;
3-box; IVR domain; Kelch domain.
```

Synthetic mRNA Architecture

The synthetic mRNA of the present invention are distinguished from wild type mRNA in their functional and/or structural design features which serve to, as evidenced herein, overcome existing problems of effective polypeptide production using nucleic acid-based therapeutics.

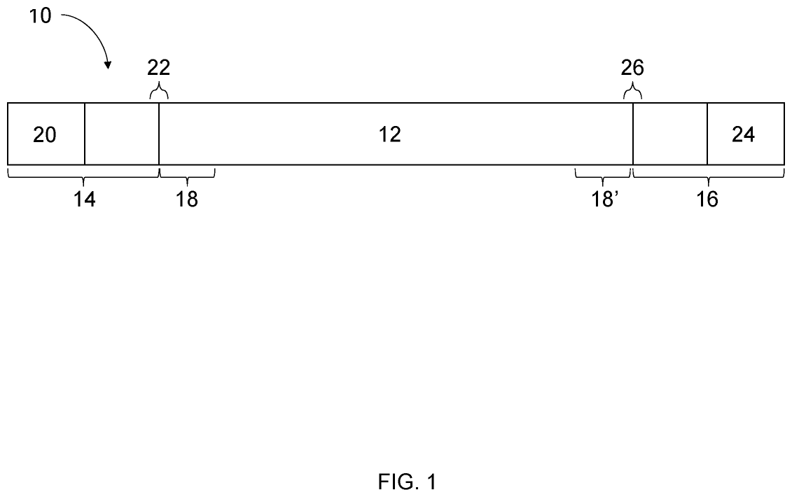

FIG. 1 shows a representative polynucleotide synthetic mRNA 10 of the present invention. As used herein, the term “synthetic mRNA” may also be referred to as “primary construct” or “primary mRNA construct”, all of which refer to a polynucleotide transcript which encodes the KEAP1 protein or a fragment thereof and which retains sufficient structural and/or chemical features to allow the polypeptide of interest encoded therein to be translated and provide a KEAP1 protein or a fragment thereof which is operable to bind to and inhibit release of Nuclear factor erythroid 2-related factor 2 (Nrf2), and presents the Nrt2 for ubiquitination and subsequent degradation. Primary constructs may be polynucleotides of the invention. When structurally or chemically modified, the primary construct may be referred to as an mmRNA, or as a synthetic mRNA.

Referring to FIG. 1, synthetic mRNA 10 here contains a first region of linked nucleotides 12 that is flanked by a first flanking region 14 and a second flaking region 16. As used herein, the “first region” 12 may be referred to as a “coding region” or “region encoding”, the “open reading frame”, “ORF”, or simply the “first region.” This first region may include, but is not limited to, the encoded KEAP1 protein or a fragment thereof of interest. The KEAP1 protein or a fragment thereof of interest may comprise at its 5′ terminus one or more additional sequences or tag, which may serve as a detection/purification tag, and/or as signal sequences encoded by a sequence encoded by region 18. The KEAP1 protein or a fragment thereof of interest may comprise at its 3′ terminus one or more additional sequences or tag, which may serve as a detection/purification tag, and/or as signal sequences encoded by a sequence encoded by region 18′. The flanking region 14 may comprise a region of linked nucleotides comprising one or more complete or incomplete 5′ UTRs sequences. The flanking region 14 may also comprise a 5′ terminal cap 20. The second flanking region 16 may comprise a region of linked nucleotides comprising one or more complete or incomplete 3′ UTRs. The flanking region 16 may also comprise a 3′ tailing sequence 24.

Bridging the 5′ terminus of the first region 12 and the first flanking region 14 is a first operational region 22. Traditionally this operational region comprises a Start codon. The operational region may alternatively comprise any translation initiation sequence or signal including a Start codon.

Bridging the 3′ terminus of the first region 12 and the second flanking region 16 is a second operational region 26. Traditionally this operational region comprises a Stop codon. The operational region may alternatively comprise any translation initiation sequence or signal including a Stop codon. According to the present invention, multiple serial stop codons may also be used.

Generally, the shortest length of the first region 12 of the synthetic mRNA/primary construct 10 of the present invention can be the length of a nucleic acid sequence that is sufficient to encode for KEAP1 protein or a fragment thereof which is operable to bind to and inhibit release of Nuclear factor erythroid 2-related factor 2 (Nrt2), and presents the Nrt2 for ubiquitination and subsequent degradation. According to an embodiment, the length of the nucleotide sequence may correspond to the length of a nucleotide sequence encoding for a KEAP1 protein according to SEQ ID NO: 1. For example, according to an embodiment, the nucleotide sequence encoding for a KEAP1 protein may be the nucleotide sequence according to SEQ ID NO: 2. In another embodiment, the length of the nucleotide sequence may correspond to the length of a nucleotide sequence encoding for the fusion of the KEAP1 protein domains which are responsible for binding to and inhibiting release of Nrf2, and presents the Nrt2 for ubiquitination and subsequent degradation. For example, according to an embodiment, the nucleotide sequence may encode for a fusion of a BTB domain of KEAP1 (e.g. comprising SEQ ID NO: 3) with a Kelch domain of KEAP1 (e.g. comprising SEQ ID NO: 5), which is operable to bind to and inhibit release of Nrt2, and presents the Nrt2 for ubiquitination and subsequent degradation. According to another embodiment, the BTB domain of KEAP1 may be functionally linked to the Kelch domain of KEAP1 via a peptide linker. For example, according to an embodiment, the nucleotide sequence may also encode a peptide linker which functionally links the BTB domain of KEAP1 and the Kelch domain of KEAP1. In embodiments, the peptide linker may comprises about 3 to about 40 amino acid residues. For example, in embodiments, the peptide linker may comprises the amino acid sequence (GGGGS)ₙ or (GGGS)ₙ wherein n≥1. For example, in embodiments, the peptide linker may comprise the amino acid sequence of SEQ ID NO: 7.

According to the present invention, the first and second flanking regions (14, 16, respectively) may range independently from 15 to 1000 nucleotides in length (e.g., greater than 30, 40, 45, 50, 55, 60, 70, 80, 90, 100, 120, 140, 160, 180, 200, 250, 300, 350, 400, 450, 500, 600, 700, 800, and 900 nucleotides or at least 30, 40, 45, 50, 55, 60, 70, 80, 90, 100, 120, 140, 160, 180, 200, 250, 300, 350, 400, 450, 500, 600, 700, 800, 900, and 1,000 nucleotides).

According to the present invention, the tailing sequence 24 may range from absent to 500 nucleotides in length (e.g., at least 60, 70, 80, 90, 100, 120, 140, 160, 180, 200, 250, 300, 350, 400, 450, or 500 nucleotides). Where the tailing region 24 is a poly(A) tail (or also known as a poly(A) region), the length may be determined in units of or as a function of poly(A) Binding Protein (PABP) binding. In this embodiment, the poly(A) tail is long enough to bind at least 4 monomers of PABP. PABP monomers bind to stretches of approximately 38 nucleotides. As such, it has been observed that poly(A) tails of about 80 nucleotides and 160 nucleotides are functional.

According to the present invention, the first and second operational regions 22, 26 respectively may range from 3 to 40, e.g., 5-30, 10-20, 15, or at least 4, or 30 or fewer nucleotides in length and may comprise, in addition to a Start and/or Stop codon, one or more signal and/or restriction sequences.

### Flanking Regions: Untranslated Regions (UTRs)

Untranslated regions (UTRs) of a gene are transcribed to mRNA, but once transcribed, they are not translated. The 5′-UTR starts at the transcription start site and continues to the start codon but does not include the start codon; whereas, the 3′-UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides, synthetic mRNA, primary constructs and/or mmRNA of the present invention to enhance the stability of the molecule. The specific features can also be incorporated to ensure controlled down-regulation of the transcript in case they are misdirected to undesired organs sites.

### 5′-UTR and Translation Initiation

Natural 5′-UTRs bear features which play roles in for translation initiation. They harbor signatures like Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′-UTR also have been known to form secondary structures which are involved in elongation factor binding.

By engineering the features typically found in abundantly expressed genes of specific target organs, one can enhance the stability and protein production of the polynucleotides, synthetic mRNA, primary constructs or mmRNA of the invention. For example, introduction of 5′-UTR of liver-expressed mRNA, such as albumin, serum amyloid A, Apolipoprotein A/B/E, transferrin, alpha fetoprotein, erythropoietin, or Factor VIII, could be used to enhance expression of a nucleic acid molecule, such as a synthetic mRNA or mmRNA, in hepatic cell lines or liver. Likewise, use of 5′-UTR from other tissue-specific mRNA to improve expression in that tissue is possible for muscle (MyoD, Myosin, Myoglobin, Myogenin, Herculin), for endothelial cells (Tie-1, CD36), for myeloid cells (C/EBP, AML1, G-CSF, GM-CSF, CD11b, MSR, Fr-1, i-NOS), for leukocytes (CD45, CD18), for adipose tissue (CD36, GLUT4, ACRP30, adiponectin) and for lung epithelial cells (SP-A/B/C/D).

Other non-UTR sequences may be incorporated into the 5′- (or 3′-UTR) UTRs. For example, introns or portions of introns sequences may be incorporated into the flanking regions of the polynucleotides, synthetic mRNA, primary constructs or mmRNA of the invention. Incorporation of intronic sequences may increase protein production as well as mRNA levels.

### 3′-UTR and the AU Rich Elements

3′-UTRs are known to have stretches of Adenosines and Uridines embedded in them. These AU rich signatures are particularly prevalent in genes with high rates of turnover. Based on their sequence features and functional properties, the AU rich elements (AREs) can be separated into three classes: Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-a. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′-UTR AU rich elements (AREs) can be used to modulate the stability of polynucleotides, synthetic mRNA, primary constructs or mmRNA of the invention. When engineering specific polynucleotides, synthetic mRNA, primary constructs or mmRNA, one or more copies of an ARE can be introduced to make polynucleotides, primary constructs or mmRNA of the invention less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using polynucleotides, primary constructs or mmRNA of the invention and protein production can be assayed at various time points post-transfection. For example, cells can be transfected with different ARE-engineering molecules and by using an ELISA kit to the relevant protein and assaying protein produced at 6-hour, 12-hour, 24-hour, 48-hour, and 7-days post-transfection.

### 5′ Capping

According to the present invention, the capping region 20 may comprise a single cap or a series of nucleotides forming the cap. In this embodiment the capping region may be from 1 to 10, e.g. 2-9, 3-8, 4-7, 1-5, 5-10, or at least 2, or 10 or fewer nucleotides in length. In some embodiments, the cap is absent.

The 5′ cap structure of an mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The 5′ cap further assists the removal of 5′ proximal introns removal during mRNA splicing.

Endogenous mRNA molecules may be 5′-end capped generating a 5′-ppp-5′-triphosphate linkage between a terminal guanosine cap residue and the 5′-terminal transcribed sense nucleotide of the mRNA molecule. This 5′-guanylate cap may then be methylated to generate an N⁷-methyl-guanylate residue. The ribose sugars of the terminal and/or anteterminal transcribed nucleotides of the 5′ end of the mRNA may optionally also be 2′-O-methylated. 5-decapping through hydrolysis and cleavage of the guanylate cap structure may target a nucleic acid molecule, such as an mRNA molecule, for degradation.

Modifications to the polynucleotides, synthetic mRNAs, primary constructs, and mmRNA of the present invention may generate a non-hydrolyzable cap structure preventing decapping and thus increasing mRNA half-life. Because cap structure hydrolysis requires cleavage of 5′-ppp-5′ phosphorodiester linkages, modified nucleotides may be used during the capping reaction. For example, a Vaccinia Capping Enzyme from New England Biolabs (Ipswich, Mass.) may be used with α-thio-guanosine nucleotides according to the manufacturer's instructions to create a phosphorothioate linkage in the 5′-ppp-5′ cap. Additional modified guanosine nucleotides may be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the mRNA (as mentioned above) on the 2′-hydroxyl group of the sugar ring. Multiple distinct 5′-cap structures can be used to generate the 5′-cap of a nucleic acid molecule, such as an mRNA molecule.

Cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs may be chemically (i.e., non-enzymatically) or enzymatically synthesized and/or linked to a nucleic acid molecule.

For example, and according to embodiments, the cap may be an Anti-Reverse Cap Analog (ARCA) cap, which contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m⁷G-3′mppp-G; which may equivalently be designated 3′ O-Me-m⁷G(5′)ppp(5′)G). The 3′-0 atom of the other, unmodified, guanine becomes linked to the 5′-terminal nucleotide of the capped nucleic acid molecule (e.g. an mRNA or mmRNA). The N7- and 3′-O-methylated guanine provides the terminal moiety of the capped nucleic acid molecule (e.g. mRNA or mmRNA).

Another exemplary cap is mCAP, which is similar to ARCA but has a 2′-O-methyl group on guanosine (i.e., N7,2′-O-dimethyl-guanosine-5′-triphosphate-5′-guanosine, m⁷Gm-ppp-G).

While cap analogs allow for the concomitant capping of a nucleic acid molecule in an in vitro transcription reaction, up to 20% of transcripts can remain uncapped. This, as well as the structural differences of a cap analog from an endogenous 5′-cap structures of nucleic acids produced by the endogenous, cellular transcription machinery, may lead to reduced translational competency and reduced cellular stability.

Polynucleotides, synthetic mRNA, primary constructs and mmRNA of the invention may also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′-cap structures of the present invention are those which, among other things, have enhanced binding of cap binding proteins, increased half-life, reduced susceptibility to 5′ endonucleases and/or reduced 5′-decapping, as compared to synthetic 5′-cap structures known in the art (or to a wild-type, natural or physiological 5′-cap structure). For example, recombinant Vaccinia Virus Capping Enzyme and recombinant 2′-O-methyltransferase enzyme can create a canonical 5′-5′-triphosphate linkage between the 5′-terminal nucleotide of an mRNA and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′-cap analog structures known in the art. In embodiments, Cap structures include, but are not limited to, ⁷mG(5′)ppp(5′)N,pN2p (cap 0), ⁷mG(5′)ppp(5′)NImpNp (cap 1), and ⁷mG(5′)-ppp(5′)NImpN2mp (cap 2).

Because the polynucleotides, synthetic mRNA, primary constructs or mmRNA may be capped post-transcriptionally, and because this process is more efficient, nearly 100% of the polynucleotides, synthetic mRNA, primary constructs or mmRNA may be capped. This is in contrast to ˜80% when a cap analog is linked to an mRNA in the course of an in vitro transcription reaction.

According to the present invention, 5′ terminal caps may include endogenous caps or cap analogs. According to the present invention, a 5′ terminal cap may comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

### Viral Sequences

Additional viral sequences such as, but not limited to, the translation enhancer sequence of the barley yellow dwarf virus (BYDV-PAV), the Jaagsiekte sheep retrovirus (JSRV) and/or the Enzootic nasal tumor virus (See e.g., International Pub. No. WO2012129648) can be engineered and inserted in the 3′-UTR of the polynucleotides, synthetic mRNA primary constructs or mmRNA of the invention and can stimulate the translation of the synthetic mRNA, construct in vitro and in vivo. Transfection experiments can be conducted in relevant cell lines at and protein production can be assayed by ELISA at 12 hr, 24 hr, 48 hr, 72 hr and day 7 post-transfection.

### IRES Sequences

Further, provided are polynucleotides, synthetic mRNA primary constructs or mmRNA which may contain an internal ribosome entry site (IRES). First identified as a feature Picorna virus RNA, IRES plays an important role in initiating protein synthesis in absence of the 5′-cap structure. An IRES may act as the sole ribosome binding site, or may serve as one of multiple ribosome binding sites of an mRNA. Polynucleotides, synthetic mRNA, primary constructs or mmRNA containing more than one functional ribosome binding site may encode several peptides or polypeptides that are translated independently by the ribosomes (“multicistronic nucleic acid molecules”). When polynucleotides, primary constructs or mmRNA are provided with an IRES, further optionally provided is a second translatable region. Examples of IRES sequences that can be used according to the invention include without limitation, those from picomaviruses (e.g. FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

### Poly(A) Tails

During RNA processing, a long chain of adenine nucleotides [poly(A) tail] may be added to a polynucleotide such as a synthetic mRNA molecule in order to increase stability. Immediately after transcription, the 3′ end of the transcript may be cleaved to free a 3′ hydroxyl. Then poly(A) polymerase adds a chain of adenine nucleotides to the RNA. The process, called polyadenylation, adds a poly(A) tail that can be between, for example, approximately 100 and 250 residues long.

It has been discovered that unique poly(A) tail lengths provide certain advantages to the polynucleotides, synthetic mRNA, primary constructs or mmRNA of the present invention. Generally, the length of a poly(A) tail of the present invention is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1000, 1100, 1200, 1300, 1,400, 1500, 1600, 1700, 1800, 1900, 2000, 2500, and 3000 nucleotides). In some embodiments, the polynucleotide, synthetic mRNA, primary construct, or mmRNA includes from about 30 to about 3,000 nucleotides (e.g., from 30 to 50, from 30 to 100, from 30 to 250, from 30 to 500, from 30 to 750, from 30 to 1000, from 30 to 1500, from 30 to 2000, from 30 to 2500, from 50 to 100, from 50 to 250, from 50 to 500, from 50 to 750, from 50 to 1000, from 50 to 1500, from 50 to 2000, from 50 to 2500, from 50 to 3000, from 100 to 500, from 100 to 750, from 100 to 1000, from 100 to 1500, from 100 to 2000, from 100 to 2500, from 100 to 3000, from 500 to 750, from 500 to 1000, from 500 to 1500, from 500 to 2000, from 500 to 2500, from 500 to 3000, from 1000 to 1500, from 1000 to 2000, from 1000 to 2500, from 1000 to 3000, from 1500 to 2000, from 1500 to 2500, from 1500 to 3000, from 2000 to 3000, from 2000 to 2500, and from 2500 to 3000).

In one embodiment, the poly(A) tail is designed relative to the length of the overall polynucleotides, synthetic mRNA, primary constructs or mmRNA. This design may be based on the length of the coding region, the length of a particular feature or region (such as the first or flanking regions) or based on the length of the ultimate product expressed from the polynucleotides, synthetic mRNA, primary constructs or mmRNA.

In this context the poly(A) tail may be 10, 20, 30, 40, 50, 60, 70, 80, 90, or 100% greater in length than the polynucleotides, synthetic mRNA, primary constructs or mmRNA or feature thereof. The poly(A) tail may also be designed as a fraction of polynucleotides, primary constructs or mmRNA to which it belongs. In this context, the poly-A tail may be 10, 20, 30, 40, 50, 60, 70, 80, or 90% or more of the total length of the construct or the total length of the construct minus the poly-A tail. Further, engineered binding sites and conjugation of polynucleotides, primary constructs or mmRNA for PABP may enhance expression.

In one embodiment, the polynucleotide, synthetic mRNA, primary constructs of the present invention are designed to include a polyA-G Quartet. The G-quartet is a cyclic hydrogen bonded array of four guanine nucleotides that can be formed by G-rich sequences in both DNA and RNA. In this embodiment, the G-quartet is incorporated at the end of the poly(A) tail. The resultant mmRNA construct is assayed for stability, protein production and other parameters including half-life at various time points. It has been discovered that the polyA-G quartet results in protein production equivalent to at least 75% of that seen using a poly(A) tail of 120 nucleotides alone.

### Modifications

Herein, in a polynucleotide (such as a primary construct or an mRNA molecule), the terms “modification” or, as appropriate, “modified” refer to modification with respect to A, G, U or C ribonucleotides. Generally, herein, these terms are not intended to refer to the ribonucleotide modifications in naturally occurring 5′-terminal mRNA cap moieties.

The modifications may be various distinct modifications. In some embodiments, the coding region, the flanking regions and/or the terminal regions may contain one, two, or more (optionally different) nucleoside or nucleotide modifications. In some embodiments, a modified polynucleotide, synthetic mRNA, primary construct, or mmRNA introduced to a cell may exhibit reduced degradation in the cell, as compared to an unmodified polynucleotide, synthetic mRNA, primary construct, or mmRNA.

The polynucleotides, synthetic mRNA, primary constructs, and mmRNA can include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications according to the present invention may be modifications of ribonucleic acids (RNAs).

In embodiments, synthetic mRNA of the present invention may comprise nucleotides comprising a modified uracil, a modified cytosine, a modified adenine, a modified guanine, or combinations thereof. The modified uracil may be N1-methyl-pseudouridine, pseudouridine, or a combination thereof. The modified uracil may be 5-methoxy-uracil. The modified cytidine may be 5-methyl-cytidine.

In some embodiments, at least 25% of the cytosines are replaced by a modified cytosine, (e.g., at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%).

In some embodiments, at least 25% of the uracils are replaced by a modified uracil, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%).

In some embodiments, at least 25% of the cytosines are replaced by a modified cytosine, and at least 25% of the uracils are replaced by a compound of Formula (b1)-(b9) (e.g., at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%).

Design and Synthesis of Synthetic mRNA

Polynucleotides, synthetic mRNA, primary constructs or mmRNA for use in accordance with the invention may be prepared according to any available technique including, but not limited to chemical synthesis, enzymatic synthesis, which is generally termed in vitro transcription (IVT) or enzymatic or chemical cleavage of a longer precursor, etc. Methods of synthesizing RNAs are known in the art (see, e.g., Gait, M. J. (ed.) Oligonucleotide synthesis: a practical approach, Oxford [Oxfordshire], Washington, D.C.: IRL Press, 1984; and Herdewijn, P. (ed.) Oligonucleotide synthesis: methods and applications, Methods in Molecular Biology, v. 288 (Clifton, N.J.) Totowa, N.J.: Humana Press, 2005; both of which are incorporated herein by reference).

The process of design and synthesis of the primary constructs of the invention generally includes the steps of gene construction, mRNA production (either with or without modifications) and purification. In the enzymatic synthesis method, a target polynucleotide sequence encoding the KEAP1 protein or a fragment thereof of interest is first selected for incorporation into a vector which will be amplified to produce a cDNA template. Optionally, the target polynucleotide sequence and/or any flanking sequences may be codon optimized. The cDNA template is then used to produce mRNA through in vitro transcription (IVT). After production, the mRNA may undergo purification and clean-up processes. The steps of which are provided in more detail below. Gene Construction

The step of gene construction may include, but is not limited to gene synthesis, vector amplification, plasmid purification, plasmid linearization and clean-up, and cDNA template synthesis and clean-up.

### Gene Synthesis

Once a polypeptide of interest, or target, is selected for production, a primary construct is designed. Within the primary construct, a first region of linked nucleosides encoding the polypeptide of interest may be constructed using an open reading frame (ORF) of a selected nucleic acid (DNA or RNA) transcript. The ORF may comprise the wild type ORF, an isoform, variant or a fragment thereof. As used herein, an “open reading frame” or “ORF” is meant to refer to a nucleic acid sequence (DNA or RNA) which is capable of encoding the KEAP1 protein or a fragment thereof of interest. ORFs often begin with the start codon, ATG and end with a nonsense or termination codon or signal.

Further, the nucleotide sequence of the first region may be codon optimized. Codon optimization methods are known in the art and may be useful in efforts to achieve one or more of several goals. These goals include to match codon frequencies in target and host organisms to ensure proper folding, bias GC content to increase mRNA stability or reduce secondary structures, minimize tandem repeat codons or base runs that may impair gene construction or expression, customize transcriptional and translational control regions, insert or remove protein trafficking sequences, remove/add post translation modification sites in encoded protein (e.g. glycosylation sites), add, remove or shuffle protein domains, insert or delete restriction sites, modify ribosome binding sites and mRNA degradation sites, to adjust translational rates to allow the various domains of the protein to fold property, or to reduce or eliminate problem secondary structures within the mRNA. Codon optimization tools, algorithms and services are known in the art, non-limiting examples include services from GeneArt (Life Technologies), DNA2.0 (Menlo Park Calif.) and/or proprietary methods. In one embodiment, the ORF sequence is optimized using optimization algorithms. Codon options for each amino acid are given in Table 1.

TABLE 1
Codon Options

| Amino Acid | Single letter code | Codon Options |
|---|---|---|
| Isoleucine | I | ATT, ATC, ATA |
| Leucine | L | CTT, CTC, CTA, CTG, TTA, TTG |
| Valine | V | GTT, GTC, GTA, GTG |
| Phenylalanine | F | TTT, TTC |
| Methionine | M | ATG |
| Cysteine | C | TGT, TGC |
| Alanine | A | GCT, GCC, GCA, GCG |
| Glycine | G | GGT, GGC, GGA, GGG |
| Proline | P | CCT, CCC, CCA, CCG |
| Threonine | T | ACT, ACC, ACA, ACG |
| Serine | S | TCT, TCC, TCA, TCG, AGT, AGC |
| Tyrosine | Y | TAT, TAC |
| Tryptophan | W | TGG |
| Glutamine | Q | CAA, CAG |
| Asparagine | N | AAT, AAC |
| Histidine | H | CAT, CAC |
| Glutamic acid | E | GAA, GAG |
| Aspartic acid | D | GAT, GAC |
| Lysine | K | AAA, AAG |
| Arginine | R | CGT, CGC, CGA, CGG, AGA, AGG |
| Selenocysteine | Sec | UGA in mRNA in |
| | | presence of Selenocystein |
| | | insertion element (SECIS) |
| Stop codons | Stop | TAA, TAG, TGA |

Features, which may be considered beneficial in some embodiments of the present invention, may be encoded by the primary construct and may flank the ORF as a first or second flanking region. The flanking regions may be incorporated into the primary construct before and/or after optimization of the ORF. It is not required that a primary construct contain both a 5′ and 3′ flanking region. Examples of such features include, but are not limited to, untranslated regions (UTRs), Kozak sequences, an oligo(dT) sequence, and detectable tags and may include multiple cloning sites which may have Xbal recognition.

In some embodiments, a 5′-UTR and/or a 3′-UTR may be provided as flanking regions. Multiple 5′- or 3′-UTRs may be included in the flanking regions and may be the same or of different sequences. Any portion of the flanking regions, including none, may be codon optimized and any may independently contain one or more different structural or chemical modifications, before and/or after codon optimization. Combinations of features may be included in the first and second flanking regions and may be contained within other features. For example, the ORF may be flanked by a 5′-UTR which may contain a strong Kozak translational initiation signal and/or a 3′-UTR which may include an oligo(dT) sequence for templated addition of a poly(A) tail. 5′-UTR may comprise a first polynucleotide fragment and a second polynucleotide fragment from the same and/or different genes such as the 5′-UTRs described in US Patent Application Publication No. 20100293625, herein incorporated by reference in its entirety.

It should be understood that any UTR from any gene may be incorporated into the respective first or second flanking region 14, 16 respectively, of the synthetic mRNA or primary construct. Furthermore, multiple wild-type UTRs of any known gene may be utilized. It is also within the scope of the present invention to provide artificial UTRs which are not variants of wild type genes. These UTRs or portions thereof may be placed in the same orientation as in the transcript from which they were selected or may be altered in orientation or location. Hence a 5′- or 3′-UTR may be inverted, shortened, lengthened, made chimeric with one or more other 5′-UTRs or 3′-UTRs. As used herein, the term “altered” as it relates to a UTR sequence, means that the UTR has been changed in some way in relation to a reference sequence. For example, a 3′- or 5′-UTR may be altered relative to a wild type or native UTR by the change in orientation or location as taught above or may be altered by the inclusion of additional nucleotides, deletion of nucleotides, swapping or transposition of nucleotides. Any of these changes producing an “altered” UTR (whether 3′- or 5′-) comprise a variant UTR.

In one embodiment, a double, triple or quadruple UTR such as a 5′- or 3′-UTR may be used. As used herein, a “double” UTR is one in which two copies of the same UTR are encoded either in series or substantially in series. For example, a double beta-globin 3′-UTR may be used as described in US Patent publication 20100129877, the contents of which are incorporated herein by reference in its entirety.

It is also within the scope of the present invention to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In one embodiment, flanking regions are selected from a family of transcripts whose proteins share a common function, structure, feature of property. For example, polypeptides of interest may belong to a family of proteins which are expressed in a particular cell, tissue or at some time during development. The UTRs from any of these genes may be swapped for any other UTR of the same or different family of proteins to create a new chimeric primary transcript. As used herein, a “family of proteins” is used in the broadest sense to refer to a group of two or more polypeptides of interest which share at least one function, structure, feature, localization, origin, or expression pattern.

After optimization (if desired), the primary construct components are reconstituted and transformed into a vector such as, but not limited to, plasmids, viruses, cosmids, and artificial chromosomes. For example, the optimized construct may be reconstituted and transformed into chemically competent E. coli, yeast, neurospora, maize, drosophila, etc. where high copy plasmid-like or chromosome structures occur by methods described herein.

The untranslated region may also include translation enhancer elements (TEE). As a non-limiting example, the TEE may include those described in US Application No. 20090226470, herein incorporated by reference in its entirety, and those known in the art.

### Stop Codons

In one embodiment, the synthetic mRNA or primary constructs of the present invention may include at least two stop codons before the 3′-UTR. The stop codon may be selected from TGA, TAA and TAG. In one embodiment, the synthetic mRNA or primary constructs of the present invention include the stop codon TGA and one additional stop codon. In a further embodiment the addition stop codon may be TAA. In another embodiment, the synthetic mRNA or the primary constructs of the present invention include three stop codons.

### Vector Amplification

The vector containing the synthetic mRNA or primary construct is then amplified and the plasmid isolated and purified using methods known in the art such as, but not limited to, a maxi prep using the Invitrogen PURELINK™ HiPure Maxiprep Kit (Carlsbad, Calif.).

### Plasmid Linearization

The plasmid may then be linearized using methods known in the art such as, but not limited to, the use of restriction enzymes and buffers. The linearization reaction may be purified using methods including, for example Invitrogen's PURELINK™ PCR Micro Kit (Carlsbad, Calif.), and HPLC based purification methods such as, but not limited to, strong anion exchange HPLC, weak anion exchange HPLC, reverse phase HPLC (RP-HPLC), and hydrophobic interaction HPLC (HIC-HPLC) and Invitrogen's standard PURELINK™ PCR Kit (Carlsbad, Calif.). The purification method may be modified depending on the size of the linearization reaction which was conducted. The linearized plasmid is then used to generate cDNA for in vitro transcription (IVT) reactions.

cDNA Template Synthesis

A cDNA template may be synthesized by having a linearized plasmid undergo polymerase chain reaction (PCR). It should be understood that that primer-probe design for any amplification is within the skill of those in the art. Probes may also contain chemically modified bases to increase base-pairing fidelity to the target molecule and base-pairing strength. Such modifications may include 5-methyl-Cytidine, 2,6-di-amino-purine, 2′-fluoro, phosphoro-thioate, or locked nucleic acids.

In one embodiment, the cDNA may be submitted for sequencing analysis before undergoing transcription.

mRNA Production

The process of mRNA, synthetic mRNA or mmRNA production may include, but is not limited to, in vitro transcription, cDNA template removal and RNA clean-up, and mRNA capping and/or tailing reactions.

### In Vitro Transcription

The cDNA produced in the previous step may be transcribed using an in vitro transcription (IVT) system. The system typically comprises a transcription buffer, nucleotide triphosphates (NTPs), an RNase inhibitor and a polymerase. The NTPs may be manufactured in house, may be selected from a supplier, or may be synthesized as described herein. The NTPs may be selected from, but are not limited to, those described herein including natural and unnatural (modified) NTPs. The polymerase may be selected from, but is not limited to, T7 RNA polymerase, T3 RNA polymerase and mutant polymerases such as, but not limited to, polymerases able to incorporate modified nucleic acids.

### RNA Polymerases

Any number of RNA polymerases or variants may be used in the design of the primary constructs of the present invention.

RNA polymerases may be modified by inserting or deleting amino acids of the RNA polymerase sequence. As a non-limiting example, the RNA polymerase may be modified to exhibit an increased ability to incorporate a 2′-modified nucleotide triphosphate compared to an unmodified RNA polymerase (see International Publication WO2008078180 and U.S. Pat. No. 8,101,385; herein incorporated by reference in their entireties).

Variants may be obtained by evolving an RNA polymerase, optimizing the RNA polymerase amino acid and/or nucleic acid sequence and/or by using other methods known in the art. As a non-limiting example, T7 RNA polymerase variants may be evolved using the continuous directed evolution system set out by Esvelt et al. (Nature (2011) 472(7344):499-503; herein incorporated by reference in its entirety) where clones of T7 RNA polymerase may encode at least one mutation such as, but not limited to, lysine at position 93 substituted for threonine (K93T), 14M, A7T, E63V, V64D, A65E, D66Y, T76N, C125R, S128R, A136T, N165S, G175R, H176L, Y178H, F182L, L196F, G198V, D208Y, E222K, S228A, Q239R, T243N, G259D, M2671, G280C, H300R, D351A, A354S, E356D, L360P, A383V, Y385C, D388Y, S397R, M401T, N410S, K450R, P451T, G452V, E484A, H523L, H524N, G542V, E565K, K577E, K577M, N601S, S684Y, L6991, K713E, N748D, Q754R, E775K, A827V, D851N or L864F. As another non-limiting example, T7 RNA polymerase variants may encode at least mutation as described in U.S. Pub. Nos. 20100120024 and 20070117112; herein incorporated by reference in their entireties. Variants of RNA polymerase may also include, but are not limited to, substitutional variants, conservative amino acid substitution, insertional variants, deletional variants and/or covalent derivatives.

In one embodiment, the synthetic mRNA or primary construct may be designed to be recognized by the wild type or variant RNA polymerases. In doing so, the primary construct may be modified to contain sites or regions of sequence changes from the wild type or parent primary construct.

In one embodiment, the synthetic mRNA or primary construct may be designed to include at least one substitution and/or insertion upstream of an RNA polymerase binding or recognition site, downstream of the RNA polymerase binding or recognition site, upstream of the TATA box sequence, downstream of the TATA box sequence of the primary construct but upstream of the coding region of the primary construct, within the 5′-UTR, before the 5′-UTR and/or after the 5′-UTR.

In one embodiment, the 5′-UTR of the primary construct may be replaced by the insertion of at least one region and/or string of nucleotides of the same base. The region and/or string of nucleotides may include, but is not limited to, at least 3, at least 4, at least 5, at least 6, at least 7 or at least 8 nucleotides and the nucleotides may be natural and/or unnatural. As a non-limiting example, the group of nucleotides may include 5-8 adenine, cytosine, thymine, a string of any of the other nucleotides disclosed herein and/or combinations thereof.

In one embodiment, the 5′-UTR of the primary construct may be replaced by the insertion of at least two regions and/or strings of nucleotides of two different bases such as, but not limited to, adenine, cytosine, thymine, any of the other nucleotides disclosed herein and/or combinations thereof. For example, the 5′-UTR may be replaced by inserting 5-8 adenine bases followed by the insertion of 5-8 cytosine bases. In another example, the 5′-UTR may be replaced by inserting 5-8 cytosine bases followed by the insertion of 5-8 adenine bases.

In one embodiment, the synthetic mRNA or primary construct may include at least one substitution and/or insertion downstream of the transcription start site which may be recognized by an RNA polymerase. As a non-limiting example, at least one substitution and/or insertion may occur downstream the transcription start site by substituting at least one nucleic acid in the region just downstream of the transcription start site (such as, but not limited to, +1 to +6). Changes to region of nucleotides just downstream of the transcription start site may affect initiation rates, increase apparent nucleotide triphosphate (NTP) reaction constant values, and increase the dissociation of short transcripts from the transcription complex curing initial transcription (Brieba et al, Biochemistry (2002) 41: 5144-5149; herein incorporated by reference in its entirety). The modification, substitution and/or insertion of at least one nucleic acid may cause a silent mutation of the nucleic acid sequence or may cause a mutation in the amino acid sequence.

In one embodiment, the synthetic mRNA or primary construct may include the substitution of at least 1, at least 2, at least 3, at least 4, at least 5, at least 6, at least 7, at least 8, at least 9, at least 10, at least 11, at least 12 or at least 13 guanine bases downstream of the transcription start site.

In one embodiment, the primary construct may include the substitution of at least 1, at least 2, at least 3, at least 4, at least 5 or at least 6 guanine bases in the region just downstream of the transcription start site. As a non-limiting example, if the nucleotides in the region are GGGAGA the guanine bases may be substituted by at least 1, at least 2, at least 3 or at least 4 adenine nucleotides. In another non-limiting example, if the nucleotides in the region are GGGAGA the guanine bases may be substituted by at least 1, at least 2, at least 3 or at least 4 cytosine bases. In another non-limiting example, if the nucleotides in the region are GGGAGA the guanine bases may be substituted by at least 1, at least 2, at least 3 or at least 4 thymine, and/or any of the nucleotides described herein.

In one embodiment, the synthetic mRNA or primary construct may include at least one substitution and/or insertion upstream of the start codon. For the purpose of clarity, one of skill in the art would appreciate that the start codon is the first codon of the protein coding region whereas the transcription start site is the site where transcription begins. The primary construct may include, but is not limited to, at least 1, at least 2, at least 3, at least 4, at least 5, at least 6, at least 7 or at least 8 substitutions and/or insertions of nucleotide bases. The nucleotide bases may be inserted or substituted at 1, at least 1, at least 2, at least 3, at least 4 or at least 5 locations upstream of the start codon. The nucleotides inserted and/or substituted may be the same base (e.g., all A or all C or all T or all G), two different bases (e.g., A and C, A and T, or C and T), three different bases (e.g., A, C and T or A, C and T) or at least four different bases. As a non-limiting example, the guanine base upstream of the coding region in the primary construct may be substituted with adenine, cytosine, thymine, or any of the nucleotides described herein. In another non-limiting example the substitution of guanine bases in the synthetic mRNA or primary construct may be designed so as to leave one guanine base in the region downstream of the transcription start site and before the start codon (see Esvelt et al. Nature (2011) 472(7344):499-503; herein incorporated by reference in its entirety). As a non-limiting example, at least 5 nucleotides may be inserted at 1 location downstream of the transcription start site but upstream of the start codon and the at least 5 nucleotides may be the same base type.

cDNA Template Removal and Clean-Up

The cDNA template may be removed using methods known in the art such as, but not limited to, treatment with Deoxyribonuclease I (DNase 1). RNA clean-up may also include a purification method such as, but not limited to, AGENCOURT® CLEANSEQ® system from Beckman Coulter (Danvers, Mass.), HPLC based purification methods such as, but not limited to, strong anion exchange HPLC, weak anion exchange HPLC, reverse phase HPLC (RP-HPLC), and hydrophobic interaction HPLC (HIC-HPLC).

Capping and/or Tailing Reactions

The synthetic mRNA or primary construct or mmRNA may also undergo capping and/or tailing reactions. A capping reaction may be performed by methods known in the art to add a 5′-cap to the 5′ end of the synthetic mRNA or primary construct. Methods for capping include, but are not limited to, using a Vaccinia Capping enzyme (New England Biolabs, Ipswich, Mass.).

A poly(A) tailing reaction may be performed by methods known in the art, such as, but not limited to, 2′ O-methyltransferase and by methods as described herein. If the primary construct generated from cDNA does not include a poly(T), it may be beneficial to perform the poly(A)-tailing reaction before the primary construct is cleaned.

mRNA Purification

Synthetic mRNA or Primary construct or mmRNA purification may include, but is not limited to, mRNA or mmRNA clean-up, quality assurance and quality control. mRNA or mmRNA clean-up may be performed by methods known in the arts such as, but not limited to, AGENCOURT® beads (Beckman Coulter Genomics, Danvers, Mass.), poly-T beads, LNA™ oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a polynucleotide such as a “purified mRNA, synthetic mRNA or mmRNA” refers to one that is separated from at least one contaminant. As used herein, a “contaminant” is any substance which makes another unfit, impure or inferior. Thus, a purified polynucleotide (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In another embodiment, the mRNA, synthetic mRNA or mmRNA may be sequenced by methods including, but not limited to reverse-transcriptase-PCR.

In one embodiment, the mRNA, synthetic mRNA or mmRNA may be quantified using methods such as, but not limited to, ultraviolet visible spectroscopy (UV/Vis). A non-limiting example of a UV/Vis spectrometer is a NANODROP® spectrometer (ThermoFisher, Waltham, Mass.). The quantified mRNA, synthetic mRNA or mmRNA may be analyzed in order to determine if the mRNA, synthetic mRNA or mmRNA may be of proper size, check that no degradation of the mRNA, synthetic mRNA or mmRNA has occurred. Degradation of the mRNA, synthetic mRNA and/or mmRNA may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Pharmaceutical Compositions

### Formulation, Administration, Delivery and Dosing

The present invention provides polynucleotides, synthetic mRNA, primary constructs and mmRNA compositions and complexes in combination with one or more pharmaceutically acceptable excipients. Pharmaceutical compositions may optionally comprise one or more additional active substances, e.g. therapeutically and/or prophylactically active substances. General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

In some embodiments, compositions are administered to humans, human patients or subjects. For the purposes of the present disclosure, the phrase “active ingredient” generally refers to polynucleotides, synthetic mRNA, primary constructs and mmRNA to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

### Formulations

The polynucleotide, synthetic mRNA, primary construct, and mmRNA of the invention can be formulated using one or more excipients to: (1) increase stability; (2) increase cell transfection; (3) permit the sustained or delayed release (e.g., from a depot formulation of the polynucleotide, synthetic mRNA, primary construct, or mmRNA); (4) alter the biodistribution (e.g., target the polynucleotide, synthetic mRNA, primary construct, or mmRNA to specific tissues or cell types); (5) increase the translation of encoded protein in vivo; and/or (6) alter the release profile of encoded protein in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with polynucleotide, synthetic mRNA, primary construct, or mmRNA (e.g., for transplantation into a subject), hyaluronidase, nanoparticle mimics and combinations thereof. Accordingly, the formulations of the invention can include one or more excipients, each in an amount that together increases the stability of the polynucleotide, synthetic mRNA, primary construct, or mmRNA, increases cell transfection by the polynucleotide, synthetic mRNA, primary construct, or mmRNA, increases the expression of polynucleotide, synthetic mRNA, primary construct, or mmRNA encoded protein, and/or alters the release profile of polynucleotide, synthetic mRNA, primary construct, or mmRNA encoded proteins. Further, the primary construct and mmRNA of the present invention may be formulated using self-assembled nucleic acid nanoparticles.

According to a preferred embodiment, the pharmaceutical composition of the present invention may comprise a plurality of lipid nanoparticles comprising a cationic lipid, a neutral lipid, a cholesterol, and a PEG lipid. The plurality of lipid nanoparticles may have a mean particle size between 80 nm and 160 nm. The plurality of lipid nanoparticles comprises within their core the synthetic mRNA of the present invention.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient may generally be equal to the dosage of the active ingredient which would be administered to a subject and/or a convenient fraction of such a dosage including, but not limited to, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient.

In some embodiments, the formulations described herein may contain at least one synthetic mRNA or mmRNA. As a non-limiting example, the formulations may contain 1, 2, 3, 4 or 5 synthetic mRNA or mmRNA.

Pharmaceutical formulations may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

In some embodiments, the particle size of the lipid nanoparticle may be increased and/or decreased. The change in particle size may be able to help counter biological reaction such as, but not limited to, inflammation or may increase the biological effect of the modified mRNA delivered to mammals.

Pharmaceutically acceptable excipients used in the manufacture of pharmaceutical compositions include, but are not limited to, inert diluents, surface active agents and/or emulsifiers, preservatives, buffering agents, lubricating agents, and/or oils. Such excipients may optionally be included in the pharmaceutical formulations of the invention.

### Liposomes, Lipoplexes, and Lipid Nanoparticles

The polynucleotide, synthetic mRNA, primary construct, and mmRNA of the invention can be formulated using one or more liposomes, lipoplexes, or lipid nanoparticles. In one embodiment, pharmaceutical compositions of polynucleotide, synthetic mRNA, primary construct, or mmRNA include liposomes. Liposomes are artificially-prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In one embodiment, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, Pa.).

In one embodiment, pharmaceutical compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; all of which are incorporated herein in their entireties). The original manufacture method by Wheeler et al. was a detergent dialysis method, which was later improved by Jeffs et al. and is referred to as the spontaneous vesicle formation method. The liposome formulations are composed of 3 to 4 lipid components in addition to the polynucleotide, primary construct, or mmRNA. As an example a liposome can contain, but is not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. As another example, certain liposome formulations may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al.

In one embodiment, pharmaceutical compositions may include liposomes which may be formed to deliver synthetic mRNA or mmRNA according to the present invention. The synthetic mRNA or mmRNA may be encapsulated by the liposome and/or it may be contained in an aqueous core which may then be encapsulated by the liposome (see International Pub. Nos. WO2012031046, WO2012031043, WO2012030901 and WO2012006378; each of which is herein incorporated by reference in their entirety). In another embodiment, the synthetic mRNA or mmRNA which may encode an immunogen may be formulated in a cationic oil-in-water emulsion where the emulsion particle comprises an oil core and a cationic lipid which can interact with the mmRNA anchoring the molecule to the emulsion particle (see International Pub. No. WO2012006380; herein incorporated by reference in its entirety). In yet another embodiment, the lipid formulation may include at least cationic lipid, a lipid which may enhance transfection and a least one lipid which contains a hydrophilic head group linked to a lipid moiety (International Pub. No. WO2011076807 and U.S. Pub. No. 20110200582; each of which is herein incorporated by reference in their entirety). In another embodiment, the polynucleotides, synthetic mRNA, primary constructs and/or mmRNA encoding an immunogen may be formulated in a lipid vesicle which may have crosslinks between functionalized lipid bilayers (see U.S. Pub. No. 20120177724, herein incorporated by reference in its entirety).

In one embodiment, the polynucleotides, synthetic mRNA, primary constructs and/or mmRNA may be formulated in a lipid vesicle which may have crosslinks between functionalized lipid bilayers.

In one embodiment, the polynucleotides, synthetic mRNA, primary constructs and/or mmRNA may be formulated in a liposome comprising a cationic lipid. The liposome may have a molar ratio of nitrogen atoms in the cationic lipid to the phophates in the RNA (N:P ratio) of between 1:1 and 20:1 as described in International Publication No. WO2013006825, herein incorporated by reference in its entirety. In another embodiment, the liposome may have a N:P ratio of greater than 20:1 or less than 1:1.

In one embodiment, the polynucleotides, synthetic mRNA, primary constructs and/or mmRNA may be formulated in a lipid-polycation complex. The formation of the lipid-polycation complex may be accomplished by methods known in the art and/or as described in U.S. Pub. No. 20120178702, herein incorporated by reference in its entirety. As a non-limiting example, the polycation may include a cationic peptide or a polypeptide such as, but not limited to, polylysine, polyomithine and/or polyarginine and the cationic peptides described in International Pub. No. WO2012013326; herein incorporated by reference in its entirety. In another embodiment, the polynucleotides, synthetic mRNA, primary constructs and/or mmRNA may be formulated in a lipid-polycation complex which may further include a neutral lipid such as, but not limited to, cholesterol or dioleoyl phosphatidylethanolamine (DOPE).

The liposome formulation may be influenced by, but not limited to, the selection of the cationic lipid component, the degree of cationic lipid saturation, the nature of the PEGylation, ratio of all components and biophysical parameters such as size. In one example by Semple et al. (Semple et al. Nature Biotech. 2010 28:172-176; herein incorporated by reference in its entirety), the liposome formulation was composed of 57.1% cationic lipid, 7.1% dipalmitoylphosphatidylcholine, 34.3% cholesterol, and 1.4% PEG-c-DMA. As another example, changing the composition of the cationic lipid could more effectively deliver siRNA to various antigen presenting cells (Basha et al. Mol Ther. 2011 19:2186-2200; herein incorporated by reference in its entirety).

In some embodiments, the ratio of PEG in the lipid nanoparticle (LNP) formulations may be increased or decreased and/or the carbon chain length of the PEG lipid may be modified from C14 to C18 to alter the pharmacokinetics and/or biodistribution of the LNP formulations. As a non-limiting example, LNP formulations may contain 1-5% of the lipid molar ratio of PEG-c-DOMG as compared to the cationic lipid, DSPC and cholesterol. In another embodiment the PEG-c-DOMG may be replaced with a PEG lipid such as, but not limited to, PEG-DSG (1,2-Distearoyl-sn-glycerol, methoxypolyethylene glycol) or PEG-DPG (1,2-Dipalmitoyl-sn-glycerol, methoxypolyethylene glycol). The cationic lipid may be selected from any lipid known in the art such as, but not limited to, DLin-MC3-DMA, DLin-DMA, C12-200 and DLin-KC2-DMA.

In one embodiment, the polynucleotides, synthetic mRNA, primary constructs or mmRNA may be formulated in a lipid nanoparticle such as those described in International Publication No. WO2012170930, herein incorporated by reference in its entirety.

In one embodiment, the cationic lipid may be selected from, but not limited to, a cationic lipid described in International Publication Nos. WO2012040184, WO2011153120, WO2011149733, WO2011090965, WO2011043913, WO2011022460, WO2012061259, WO2012054365, WO2012044638, WO2010080724, WO201021865 and WO2008103276, U.S. Pat. Nos. 7,893,302, 7,404,969 and 8,283,333 and US Patent Publication No. US20100036115 and US20120202871; each of which is herein incorporated by reference in their entirety. In another embodiment, the cationic lipid may be selected from, but not limited to, formula A described in International Publication Nos. WO2012040184, WO2011153120, WO2011149733, WO2011090965, WO2011043913, WO2011022460, WO2012061259, WO2012054365 and WO2012044638; each of which is herein incorporated by reference in their entirety. In yet another embodiment, the cationic lipid may be selected from, but not limited to, formula CLI-CLXXIX of International Publication No. WO2008103276, formula CLI-CLXXIX of U.S. Pat. No. 7,893,302, formula CLI-CLXXXXII of U.S. Pat. No. 7,404,969 and formula I-VI of US Patent Publication No. US20100036115; each of which is herein incorporated by reference in their entirety. As a non-limiting example, the cationic lipid may be selected from (20Z,23Z)-N,N-dimethylnonacosa-20,23-dien-10-amine, (17Z,20Z)-N,N-dimemylhexacosa-17,20-dien-9-amine, (1Z,19Z)-N5N-dimethylpentacosa-16,19-dien-8-amine, (13Z,16Z)-N,N-dimethyldocosa-13,16-dien-5-amine, (12Z,15Z)-N,N-dimethylhenicosa-12,15-dien-4-amine, (14Z,17Z)-N,N-dimethyltricosa-14,17-dien-6-amine, (15Z,18Z)-N,N-dimethyltetracosa-15,18-dien-7-amine, (18Z,21Z)-N,N-dimethylheptacosa-18,21-dien-10-amine, (15Z,18Z)-N,N-dimethyltetracosa-15,18-dien-5-amine, (14Z,17Z)-N,N-dimethyltricosa-14,17-dien-4-amine, (19Z,22Z)-N,N-dimeihyloctacosa-19,22-dien-9-amine, (18Z,21Z)-N,N-dimethylheptacosa-18,21-dien-8-amine, (17Z,20Z)-N,N-dimethylhexacosa-17,20-dien-7-amine, (16Z,19Z)-N,N-dimethylpentacosa-16,19-dien-6-amine, (22Z,25Z)-N,N-dimethylhentriaconta-22,25-dien-10-amine, (21Z,24Z)-N,N-dimethyltriaconta-21,24-dien-9-amine, (18Z)-N,N-dimetylheptacos-18-en-10-amine, (17Z)-N,N-dimethylhexacos-17-en-9-amine, (19Z,22Z)-N,N-dimethyloctacosa-19,22-dien-7-amine, N,N-dimethylheptacosan-10-amine, (20Z,23Z)-N-ethyl-N-methylnonacosa-20,23-dien-10-amine, 1-[(11Z,14Z)-1-nonylicosa-11,14-dien-1-yl]pyrrolidine, (20Z)-N,N-dimethylheptacos-20-en-10-amine, (15Z)-N,N-dimethyl eptacos-15-en-10-amine, (14Z)-N,N-dimethylnonacos-14-en-10-amine, (17Z)-N,N-dimethylnonacos-17-en-10-amine, (24Z)-N,N-dimethyltritriacont-24-en-10-amine, (20Z)-N,N-dimethylnonacos-20-en-10-amine, (22Z)-N,N-dimethylhentriacont-22-en-10-amine, (16Z)-N,N-dimethylpentacos-16-en-8-amine, (12Z,15Z)-N,N-dimethyl-2-nonylhenicosa-12,15-dien-1-amine, (13Z,16Z)-NN-dimethyl-3-nonyidocosa-13,16-dien-1-amine, N,N-dimethyl-1-[(1S,2R)-2-octylcycdopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N, N-dimethyl-21-[(1S,2R)-2-octylcyclopropyl]henicosan-10-amine, N,N-dimethyl-1-[(1S,2S)-2-{[(1R,2R)-2-pentylcyclopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octykcycopropyl]pentadecan-8-amine, R-N,N-dimethyl-1-[(9Z,12Z)-octadeca-9,12-dien-1-yloxy]-3-(octyloxy)propan-2-amine, S-N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)-N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)-N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)-N,N-dimethyl-H(1-methyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-octylcyclopropyl)octyl]oxy}-3-(octyloxy)propan-2-amine and (11E,20Z,23Z)-N,N-dimethylnonacosa-11,20,2-trien-10-amine or a pharmaceutically acceptable salt or stereoisomer thereof.

In one embodiment, the lipid may be a cleavable lipid such as those described in International Publication No. WO2012170889, herein incorporated by reference in its entirety.

In one embodiment, the cationic lipid may be synthesized by methods known in the art and/or as described in International Publication Nos. WO2012040184, WO2011153120, WO2011149733, WO2011090965, WO2011043913, WO2011022460, WO2012061259, WO2012054365, WO2012044638, WO2010080724 and WO201021865; each of which is herein incorporated by reference in their entirety.

In one embodiment, the LNP formulations of the polynucleotides, primary constructs and/or mmRNA may contain PEG-c-DOMG at 3% lipid molar ratio. In another embodiment, the LNP formulations polynucleotides, primary constructs and/or mmRNA may contain PEG-c-DOMG at 1.5% lipid molar ratio.

In one embodiment, the pharmaceutical compositions of the polynucleotides, synthetic mRNA, primary constructs and/or mmRNA may include at least one of the PEGylated lipids described in International Publication No. 2012099755, herein incorporated by reference.

In one embodiment, the LNP formulation may contain PEG-DMG 2000 (1,2-dimyristoyl-sn-glycero-3-phophoethanolamine-N-[methoxy(polyethylene glycol)-2000). In one embodiment, the LNP formulation may contain PEG-DMG 2000, a cationic lipid known in the art and at least one other component. In another embodiment, the LNP formulation may contain PEG-DMG 2000, a cationic lipid known in the art, DSPC and cholesterol. As a non-limiting example, the LNP formulation may contain PEG-DMG 2000, DLin-DMA, DSPC and cholesterol. As another non-limiting example the LNP formulation may contain PEG-DMG 2000, DLin-DMA, DSPC and cholesterol in a molar ratio of 2:40:10:48 (see e.g., Geall et al., Nonviral delivery of self-amplifying RNA vaccines, PNAS 2012; PMID: 22908294; herein incorporated by reference in its entirety). As another non-limiting example, modified RNA described herein may be formulated in a nanoparticle to be delivered by a parenteral route as described in U.S. Pub. No. 20120207845; herein incorporated by reference in its entirety.

In one embodiment, the LNP formulation may be formulated by the methods described in International Publication Nos. WO2011127255 or WO2008103276, each of which is herein incorporated by reference in their entirety. As a non-limiting example, modified RNA described herein may be encapsulated in LNP formulations as described in WO2011127255 and/or WO2008103276; each of which is herein incorporated by reference in their entirety.

In one embodiment, LNP formulations described herein may comprise a polycationic composition. As a non-limiting example, the polycationic composition may be selected from formula 1-60 of US Patent Publication No. US20050222064; herein incorporated by reference in its entirety. In another embodiment, the LNP formulations comprising a polycationic composition may be used for the delivery of the modified RNA described herein in vivo and/or in vitro.

In one embodiment, the LNP formulations described herein may additionally comprise a permeability enhancer molecule. Non-limiting permeability enhancer molecules are described in US Patent Publication No. US20050222064; herein incorporated by reference in its entirety.

In one embodiment, the pharmaceutical compositions may be formulated in liposomes such as, but not limited to, DiLa2 liposomes (Marina Biotech, Bothell, Wash.), SMARTICLES® (Marina Biotech, Bothell, Wash.), neutral DOPC (1,2-dioleoyl-sn-glycero-3-phosphocholine) based liposomes (e.g., siRNA delivery for ovarian cancer (Landen et al. Cancer Biology & Therapy 2006 5(12) 1708-1713); herein incorporated by reference in its entirety) and hyaluronan-coated liposomes (Quiet Therapeutics, Israel).

The nanoparticle formulations may be a carbohydrate nanoparticle comprising a carbohydrate carrier and a synthetic mRNA or modified nucleic acid molecule (e.g., mmRNA). As a non-limiting example, the carbohydrate carrier may include, but is not limited to, an anhydride-modified phytoglycogen or glycogen-type material, phytoglycogen octenyl succinate, phytoglycogen beta-dextrin, anhydride-modified phytoglycogen beta-dextrin. (See e.g., International Publication No. WO2012109121; herein incorporated by reference in its entirety).

Lipid nanoparticle formulations may be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and may be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it may be terminally located at the terminal end of the lipid chain. The internal ester linkage may replace any carbon in the lipid chain.

In one embodiment such formulations may also be constructed or compositions altered such that they passively or actively are directed to different cell types in vivo, including but not limited to hepatocytes, immune cells, tumor cells, endothelial cells, antigen presenting cells, and leukocytes (Akinc et al. Mol Ther. 2010 18:1357-1364; Song et al., Nat Biotechnol. 2005 23:709-717; Judge et al., J Clin Invest. 2009 119:661-673; Kaufmann et al., Microvasc Res 2010 80:286-293; Santel et al., Gene Ther 2006 13:1222-1234; Santel et al., Gene Ther 2006 13:1360-1370; Gutbier et al., Pulm Pharmacol. Ther. 2010 23:334-344; Basha et al., Mol. Ther. 2011 19:2186-2200; Fenske and Cullis, Expert Opin Drug Deliv. 2008 5:25-44; Peer et al., Science. 2008 319:627-630; Peer and Lieberman, Gene Ther. 2011 18:1127-1133; all of which are incorporated herein by reference in its entirety). One example of passive targeting of formulations to liver cells includes the DLin-DMA, DLin-KC2-DMA and DLin-MC3-DMA-based lipid nanoparticle formulations which have been shown to bind to apolipoprotein E and promote binding and uptake of these formulations into hepatocytes in vivo (Akinc et al. Mol Ther. 2010 18:1357-1364; herein incorporated by reference in its entirety). Formulations can also be selectively targeted through expression of different ligands on their surface as exemplified by, but not limited by, folate, transferrin, N-acetylgalactosamine (GalNAc), and antibody targeted approaches (Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; Peer and Lieberman, Gene Ther. 2011 18:1127-1133; all of which are incorporated herein by reference in its entirety).

### Excipients

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, Md., 2006; incorporated herein by reference in its entirety) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEENn®60], polyoxyethylene sorbitan monooleate [TWEEN®80], sorbitan monopalmitate [SPAN®40], sorbitan monostearate [SPAN®60], sorbitan tristearate [SPAN®65], glceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC® F 68, POLOXAMER®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN® II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringers solution, ethyl alcohol, etc., and/or combinations thereof.

Exemplary lubricating agents include, but are not limited to, magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

### Administration

The polynucleotides, synthetic mRNA, primary constructs or mmRNA of the present invention may be administered by any route which results in a therapeutically effective outcome. These include, but are not limited to enteral, gastroenterol, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intravenous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. In specific embodiments, compositions may be administered in a way which allows them cross the blood-brain barrier, vascular barrier, or other epithelial barrier. Non-limiting routes of administration for the polynucleotides, synthetic mRNA, primary constructs or mmRNA of the present invention are described below.

### Parenteral and Injectable Administration

Liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

### Rectal and Vaginal Administration

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

### Oral Administration

Liquid dosage forms for oral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates, cyclodextrins, polymers, and/or combinations thereof.

Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

### Topical or Transdermal Administration

As described herein, compositions containing the polynucleotides, primary constructs or mmRNA of the invention may be formulated for administration topically. The skin may be an ideal target site for delivery as it is readily accessible. Gene expression may be restricted not only to the skin, potentially avoiding nonspecific toxicity, but also to specific layers and cell types within the skin.

The site of cutaneous expression of the delivered compositions will depend on the route of nucleic acid delivery. Three routes are commonly considered to deliver polynucleotides, primary constructs or mmRNA to the skin: (i) topical application (e.g. for local/regional treatment and/or cosmetic applications); (ii) intradermal injection (e.g. for local/regional treatment and/or cosmetic applications); and (iii) systemic delivery (e.g. for treatment of dermatologic diseases that affect both cutaneous and extracutaneous regions). Polynucleotides, primary constructs or mmRNA can be delivered to the skin by several different approaches known in the art. Most topical delivery approaches have been shown to work for delivery of DNA, such as but not limited to, topical application of non-cationic liposome-DNA complex, cationic liposome-DNA complex, particle-mediated (gene gun), puncture-mediated gene transfections, and viral delivery approaches. After delivery of the nucleic acid, gene products have been detected in a number of different skin cell types, including, but not limited to, basal keratinocytes, sebaceous gland cells, dermal fibroblasts and dermal macrophages.

In one embodiment, the invention provides for a variety of dressings (e.g., wound dressings) or bandages (e.g., adhesive bandages) for conveniently and/or effectively carrying out methods of the present invention. Typically dressing or bandages may comprise sufficient amounts of pharmaceutical compositions and/or polynucleotides, primary constructs or mmRNA described herein to allow a user to perform multiple treatments of a subject(s).

In one embodiment, the invention provides for the polynucleotides, primary constructs or mmRNA compositions to be delivered in more than one injection.

In one embodiment, before topical and/or transdermal administration at least one area of tissue, such as skin, may be subjected to a device and/or solution which may increase permeability. In one embodiment, the tissue may be subjected to an abrasion device to increase the permeability of the skin (see U.S. Patent Publication No. 20080275468, herein incorporated by reference in its entirety). In another embodiment, the tissue may be subjected to an ultrasound enhancement device. An ultrasound enhancement device may include, but is not limited to, the devices described in U.S. Publication No. 20040236268 and U.S. Pat. Nos. 6,491,657 and 6,234,990; each of which are herein incorporated by reference in their entireties. Methods of enhancing the permeability of tissue are described in U.S. Publication Nos. 20040171980 and 20040236268 and U.S. Pat. No. 6,190,315; each of which are herein incorporated by reference in their entireties.

In one embodiment, a device may be used to increase permeability of tissue before delivering formulations of modified mRNA described herein. The permeability of skin may be measured by methods known in the art and/or described in U.S. Pat. No. 6,190,315, herein incorporated by reference in its entirety. As a non-limiting example, a modified mRNA formulation may be delivered by the drug delivery methods described in U.S. Pat. No. 6,190,315, herein incorporated by reference in its entirety.

In another non-limiting example tissue may be treated with a eutectic mixture of local anesthetics (EMLA) cream before, during and/or after the tissue may be subjected to a device which may increase permeability. Katz et al. (Anesth Analg (2004); 98:371-76; herein incorporated by reference in its entirety) showed that using the EMLA cream in combination with a low energy, an onset of superficial cutaneous analgesia was seen as fast as 5 minutes after a pretreatment with a low energy ultrasound.

In one embodiment, enhancers may be applied to the tissue before, during, and/or after the tissue has been treated to increase permeability. Enhancers include, but are not limited to, transport enhancers, physical enhancers, and cavitation enhancers. Non-limiting examples of enhancers are described in U.S. Pat. No. 6,190,315, herein incorporated by reference in its entirety.

In one embodiment, a device may be used to increase permeability of tissue before delivering formulations of modified mRNA described herein, which may further contain a substance that invokes an immune response. In another non-limiting example, a formulation containing a substance to invoke an immune response may be delivered by the methods described in U.S. Publication Nos. 20040171980 and 20040236268; each of which are herein incorporated by reference in their entireties.

Dosage forms for topical and/or transdermal administration of a composition may include ointments, pastes, creams, lotions, gels, powders, solutions, sprays, inhalants and/or patches. Generally, an active ingredient is admixed under sterile conditions with a pharmaceutically acceptable excipient and/or any needed preservatives and/or buffers as may be required.

Additionally, the present invention contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions.

Topically-administrable formulations may, for example, comprise from about 0.1% to about 10% (w/w) active ingredient, although the concentration of active ingredient may be as high as the solubility limit of the active ingredient in the solvent. Formulations for topical administration may further comprise one or more of the additional ingredients described herein.

### Depot Administration

As described herein, in some embodiments, the composition is formulated in depots for extended release. Generally, a specific organ or tissue (a “target tissue”) is targeted for administration.

In some aspects of the invention, the polynucleotides, synthetic mRNA, primary constructs or mmRNA are spatially retained within or proximal to a target tissue. Provided are method of providing a composition to a target tissue of a mammalian subject by contacting the target tissue (which contains one or more target cells) with the composition under conditions such that the composition, in particular the nucleic acid component(s) of the composition, is substantially retained in the target tissue, meaning that at least 10, 20, 30, 40, 50, 60, 70, 80, 85, 90, 95, 96, 97, 98, 99, 99.9, 99.99 or greater than 99.99% of the composition is retained in the target tissue. Advantageously, retention is determined by measuring the amount of the nucleic acid present in the composition that enters one or more target cells. For example, at least 1, 5, 10, 20, 30, 40, 50, 60, 70, 80, 85, 90, 95, 96, 97, 98, 99, 99.9, 99.99 or greater than 99.99% of the nucleic acids administered to the subject are present intracellularly at a period of time following administration. For example, intramuscular injection to a mammalian subject is performed using an aqueous composition containing a ribonucleic acid and a transfection reagent, and retention of the composition is determined by measuring the amount of the ribonucleic acid present in the muscle cells.

Aspects of the invention are directed to methods of providing a composition to a target tissue of a mammalian subject, by contacting the target tissue (containing one or more target cells) with the composition under conditions such that the composition is substantially retained in the target tissue. The composition contains an effective amount of a polynucleotides, synthetic mRNA, primary constructs or mmRNA such that the polypeptide of interest is produced in at least one target cell. The compositions generally contain a cell penetration agent, although “naked” nucleic acid (such as nucleic acids without a cell penetration agent or other agent) is also contemplated, and a pharmaceutically acceptable carrier.

In some circumstances, the amount of a protein produced by cells in a tissue is desirably increased. Preferably, this increase in protein production is spatially restricted to cells within the target tissue. Thus, provided are methods of increasing production of a protein of interest in a tissue of a mammalian subject. A composition is provided that contains polynucleotides, synthetic mRNA, primary constructs or mmRNA characterized in that a unit quantity of composition has been determined to produce the polypeptide of interest in a substantial percentage of cells contained within a predetermined volume of the target tissue.

In some embodiments, the composition includes a plurality of different polynucleotides, synthetic mRNA, primary constructs or mmRNA, where one or more than one of the polynucleotides, synthetic mRNA, primary constructs or mmRNA encodes a polypeptide of interest. Optionally, the composition also contains a cell penetration agent to assist in the intracellular delivery of the composition. A determination is made of the dose of the composition required to produce the polypeptide of interest in a substantial percentage of cells contained within the predetermined volume of the target tissue (generally, without inducing significant production of the polypeptide of interest in tissue adjacent to the predetermined volume, or distally to the target tissue). Subsequent to this determination, the determined dose is introduced directly into the tissue of the mammalian subject.

In one embodiment, the invention provides for the polynucleotides, synthetic mRNA, primary constructs or mmRNA to be delivered in more than one injection or by split dose injections.

In one embodiment, the invention may be retained near target tissue using a small disposable drug reservoir, patch pump or osmotic pump. Non-limiting examples of patch pumps include those manufactured and/or sold by BD® (Franklin Lakes, N.J.), Insulet Corporation (Bedford, Mass.), SteadyMed Therapeutics (San Francisco, Calif.), Medtronic (Minneapolis, Minn.) (e.g., MiniMed), UniLife (York, Pa.), Valeritas (Bridgewater, N.J.), and SpringLeaf Therapeutics (Boston, Mass.). A non-limiting example of an osmotic pump include those manufactured by DURECT® (Cupertino, Calif.) (e.g., DUROS® and ALZET®).

### Pulmonary Administration

A pharmaceutical composition may be prepared, packaged, and/or sold in a formulation suitable for pulmonary administration via the buccal cavity. Such a formulation may comprise dry particles which comprise the active ingredient and which have a diameter in the range from about 0.5 nm to about 7 nm or from about 1 nm to about 6 nm. Such compositions are suitably in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 18° C. at atmospheric pressure. Generally the propellant may constitute 50% to 99.9% (w/w) of the composition, and active ingredient may constitute 0.1% to 20% (w/w) of the composition. A propellant may further comprise additional ingredients such as a liquid non-ionic and/or solid anionic surfactant and/or a solid diluent (which may have a particle size of the same order as particles comprising the active ingredient).

As a non-limiting example, the polynucleotides, synthetic mRNA, primary constructs and/or mmRNA described herein may be formulated for pulmonary delivery by the methods described in U.S. Pat. No. 8,257,685; herein incorporated by reference in its entirety.

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

### Intranasal, Nasal and Buccal Administration

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

### Ophthalmic Administration

A pharmaceutical composition may be prepared, packaged, and/or sold in a formulation suitable for ophthalmic administration. Such formulations may, for example, be in the form of eye drops including, for example, a 0.1/1.0% (w/w) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Ear drops and/or eye drops are contemplated as being within the scope of this invention. A multilayer thin film device may be prepared to contain a pharmaceutical composition for delivery to the eye and/or surrounding tissue.

### Combinations

The polynucleotides, primary constructs or mmRNA may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, or imaging compositions in combination with agents that may improve their bioavailability, reduce and/or modify their metabolism, inhibit their excretion, and/or modify their distribution within the body.

Therefore, and according to another embodiment, there is disclosed a method for the treatment of a disease associated with KEAP1 and/or Nrf2 comprising (a) systematically administering to a patient in need thereof a therapeutically effective amount of a first therapeutic agent comprising the pharmaceutical composition of the present invention comprising the synthetic mRNA of the present invention; and (b) administering to the patient in need thereof a therapeutically effective amount of a second therapeutic agent. By “second therapeutic agent” it is not intended to imply that the agent is the same as the first therapeutic agent comprising the pharmaceutical composition of the present invention; the second therapeutic agent is distinct from the first therapeutic agent. According to some embodiments, the second therapeutic agent may comprise doxorubicin, cisplatin, carboplatin, docetaxel, paclitaxel, protein-bound paclitaxel, cabazitaxel gemcitabine, camptothecin, daunorubicin, idarubicin, hydoxyurea, decitabine, azacytidine, cytarabine, 5-Fluorouracil, capecitabine, irinotecan, oxaliplatin, trifluridine and tipiracil, mitoxantrone, vinorelbine, pemetrexed, leucovorin, irinotecan, folfirinox, epirubicin, estramustine, cyclophosphamide, trastuzumab, pertuzumab.

By “disease associated with KEAP1 and/or Nrf2”, the person skilled in the art would understand that these are disease associated with loss of KEAP1 function, or premature degradation of KEAP1, and/or increased Nrf2 activity. For example, KEAP1 is known to be implicated in disease such as multinodular goiter, and many forms of cancer. Nrf2 may be involved in renal injury, loss of kidney function, oxidative and reticulum endoplasmic stress, cell death and many forms of cancer. For example, the cancer may be a bladder cancer, a leukemia, a colon cancer, a kidney cancer, a liver cancer, a lung cancer, a pancreatic cancer, a stomach cancer, a esophageal cancer, a skin cancer, a prostate cancer, and a breast cancer.

As a non-limiting example, the nucleic acids, synthetic mRNA or mmRNA of the present invention may be used in combination with a pharmaceutical agent for the treatment of cancer or to control hyperproliferative cells.

It will further be appreciated that therapeutically, prophylactically, diagnostically, or imaging active agents utilized in combination may be administered together in a single composition or administered separately in different compositions. In general, it is expected that agents utilized in combination with be utilized at levels that do not exceed the levels at which they are utilized individually. In some embodiments, the levels utilized in combination will be lower than those utilized individually. In one embodiment, the combinations, each or together may be administered according to the split dosing regimens described herein.

### Dosing

The present invention provides methods comprising administering synthetic mRNAs or modified mRNAs and their encoded proteins or complexes in accordance with the invention to a subject in need thereof. Nucleic acids, proteins or complexes, or pharmaceutical, imaging, diagnostic, or prophylactic compositions thereof, may be administered to a subject using any amount and any route of administration effective for preventing, treating, diagnosing, or imaging a disease, disorder, and/or condition (e.g., a disease, disorder, and/or condition relating to working memory deficits). The exact amount required will vary from subject to subject, depending on the species, age, and general condition of the subject, the severity of the disease, the particular composition, its mode of administration, its mode of activity, and the like. Compositions in accordance with the invention are typically formulated in dosage unit form for ease of administration and uniformity of dosage. It will be understood, however, that the total daily usage of the compositions of the present invention may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In certain embodiments, compositions in accordance with the present invention may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used.

According to the present invention, it has been discovered that administration of synthetic mRNA or mmRNA in split-dose regimens produce higher levels of proteins in mammalian subjects. As used herein, a “split dose” is the division of single unit dose or total daily dose into two or more doses, e.g, two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose. In one embodiment, the mmRNA of the present invention are administered to a subject in split doses. The mmRNA may be formulated in buffer only or in a formulation described herein.

### Dosage Forms

A pharmaceutical composition described herein can be formulated into a dosage form described herein, such as a topical, intranasal, intratracheal, or injectable (e.g., intravenous, intraocular, intravitreal, intramuscular, intracardiac, intraperitoneal, subcutaneous).

### Liquid Dosage Forms

Liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art including, but not limited to, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. In certain embodiments for parenteral administration, compositions may be mixed with solubilizing agents such as CREMOPHOR®, alcohols, oils, modified oils, glycols, polysorbates, cyclodextrins, polymers, and/or combinations thereof.

### Injectable

Injectable preparations, for example, sterile injectable aqueous or oleaginous suspensions may be formulated according to the known art and may include suitable dispersing agents, wetting agents, and/or suspending agents. Sterile injectable preparations may be sterile injectable solutions, suspensions, and/or emulsions in nontoxic parenterally acceptable diluents and/or solvents, for example, a solution in 1,3-butanediol. Among the acceptable vehicles and solvents that may be employed include, but are not limited to, water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it may be desirable to slow the absorption of the active ingredient from subcutaneous or intramuscular injection. This may be accomplished by the use of a liquid suspension of crystalline or amorphous material with poor water solubility. The rate of absorption of the polynucleotide, synthetic mRNA, primary construct or mmRNA then depends upon its rate of dissolution which, in turn, may depend upon crystal size and crystalline form. Alternatively, delayed absorption of a parenterally administered polynucleotide, synthetic mRNA, primary construct or mmRNA may be accomplished by dissolving or suspending the polynucleotide, synthetic mRNA, primary construct or mmRNA in an oil vehicle. Injectable depot forms are made by forming microencapsule matrices of the polynucleotide, synthetic mRNA, primary construct or mmRNA in biodegradable polymers such as polylactide-polyglycolide. Depending upon the ratio of polynucleotide, synthetic mRNA, primary construct or mmRNA to polymer and the nature of the particular polymer employed, the rate of polynucleotide, synthetic mRNA, primary construct or mmRNA release can be controlled. Examples of other biodegradable polymers include, but are not limited to, poly(orthoesters) and poly(anhydrides). Depot injectable formulations may be prepared by entrapping the polynucleotide, synthetic mRNA, primary construct or mmRNA in liposomes or microemulsions which are compatible with body tissues.

### Pulmonary

Formulations described herein as being useful for pulmonary delivery may also be used for intranasal delivery of a pharmaceutical composition. Another formulation suitable for intranasal administration may be a coarse powder comprising the active ingredient and having an average particle from about 0.2 μm to 500 μm. Such a formulation may be administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, contain about 0.1% to 20% (w/w) active ingredient, where the balance may comprise an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

General considerations in the formulation and/or manufacture of pharmaceutical agents may be found, for example, in Remington: The Science and Practice of Pharmacy 21st ed., Lippincott Williams & Wilkins, 2005 (incorporated herein by reference in its entirety).

### Coatings or Shells

Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

### Properties of Pharmaceutical Compositions

The pharmaceutical compositions described herein can be characterized by one or more of bioavailability, therapeutic window and/or volume of distribution.

### Bioavailability

The polynucleotides, synthetic mRMA, primary constructs or mmRNA, when formulated into a composition with a delivery agent as described herein, can exhibit an increase in bioavailability as compared to a composition lacking a delivery agent as described herein. As used herein, the term “bioavailability” refers to the systemic availability of a given amount of polynucleotides, synthetic mRMA, primary constructs or mmRNA administered to a mammal. Bioavailability can be assessed by measuring the area under the curve (AUC) or the maximum serum or plasma concentration (Cmax) of the unchanged form of a compound following administration of the compound to a mammal. AUC is a determination of the area under the curve plotting the serum or plasma concentration of a compound along the ordinate (Y-axis) against time along the abscissa (X-axis). Generally, the AUC for a particular compound can be calculated using methods known to those of ordinary skill in the art and as described in G. S. Banker, Modern Pharmaceutics, Drugs and the Pharmaceutical Sciences, v. 72, Marcel Dekker, New York, Inc., 1996, herein incorporated by reference in its entirety.

The Cmax, value is the maximum concentration of the compound achieved in the serum or plasma of a mammal following administration of the compound to the mammal. The Cmax value of a particular compound can be measured using methods known to those of ordinary skill in the art. The phrases “increasing bioavailability” or “improving the pharmacokinetics,” as used herein mean that the systemic availability of a first polynucleotide, primary construct or mmRNA, measured as AUC, Cmax, or Cmin in a mammal is greater, when co-administered with a delivery agent as described herein, than when such co-administration does not take place. In some embodiments, the bioavailability of the polynucleotide, primary construct or mmRNA can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

### Biological Effect

In one embodiment, the biological effect of the modified mRNA delivered to the animals may be categorized by analyzing the protein expression in the animals. The protein expression may be determined from analyzing a biological sample collected from a mammal administered the modified mRNA of the present invention. In one embodiment, the expression protein encoded by the modified mRNA administered to the mammal of at least 50 μg/ml may be preferred. For example, a protein expression of 50-200 μg/ml for the protein encoded by the modified mRNA delivered to the mammal may be seen as a therapeutically effective amount of protein in the mammal.

### Kits

The invention provides a variety of kits for conveniently and/or effectively carrying out methods of the present invention. Typically kits will comprise sufficient amounts and/or numbers of components to allow a user to perform multiple treatments of a subject(s) and/or to perform multiple experiments.

In one aspect, the present invention provides kits comprising the molecules (polynucleotides, synthetic mRNA, primary constructs or mmRNA) of the invention. In one embodiment, the kit comprises one or more functional antibodies or function fragments thereof.

Said kits can be for protein production, comprising a first polynucleotide, synthetic mRNA, primary construct or mmRNA comprising a translatable region. The kit may further comprise packaging and instructions and/or a delivery agent to form a formulation composition. The delivery agent may comprise a saline, a buffered solution, a lipidoid or any delivery agent disclosed herein.

In one embodiment, the buffer solution may include sodium chloride, calcium chloride, phosphate and/or EDTA. In another embodiment, the buffer solution may include, but is not limited to, saline, saline with 2 mM calcium, 5% sucrose, 5% sucrose with 2 mM calcium, 5% Mannitol, 5% Mannitol with 2 mM calcium, Ringer's lactate, sodium chloride, sodium chloride with 2 mM calcium and mannose (See e.g., U.S. Pub. No. 20120258046; herein incorporated by reference in its entirety). In a further embodiment, the buffer solutions may be precipitated or it may be lyophilized. The amount of each component may be varied to enable consistent, reproducible higher concentration saline or simple buffer formulations. The components may also be varied in order to increase the stability of modified RNA in the buffer solution over a period of time and/or under a variety of conditions. In one aspect, the present invention provides kits for protein production, comprising: a polynucleotide, primary construct or mmRNA comprising a translatable region, provided in an amount effective to produce a desired amount of a protein encoded by the translatable region when introduced into a target cell; a second polynucleotide comprising an inhibitory nucleic acid, provided in an amount effective to substantially inhibit the innate immune response of the cell; and packaging and instructions.

In one aspect, the present invention provides kits for protein production, comprising a polynucleotide, primary construct or mmRNA comprising a translatable region, wherein the polynucleotide exhibits reduced degradation by a cellular nuclease, and packaging and instructions.

In one aspect, the present invention provides kits for protein production, comprising a polynucleotide, synthetic mRNA, primary construct or mmRNA comprising a translatable region, wherein the polynucleotide exhibits reduced degradation by a cellular nuclease, and a mammalian cell suitable for translation of the translatable region of the first nucleic acid.

The present invention will be more readily understood by referring to the following examples which are given to illustrate the invention rather than to limit its scope.

### Example 1

In Vitro Transcription of KEAP1 mRNA

Wild type human KEAP1 mRNA (mmRNA) according to the invention is made using standard laboratory methods and materials. Plasmids RC202513 and RC202189 (OriGene®) comprising the KEAP1 open reading frame (ORF) (SEQ ID NO: 2) are isolated. The plasmids are linearized with the enzyme BamH1, and purified with QIAquick™ PCR purification kit, QIAGen™, Cat: 28104) before use as a template for in vitro KEAP1 mRNA transcription. KEAP1 mRNA is then produced by in vitro transcription from the linearized plasmid using the New England BioLabs the E2060 in vitro mRNA transcription kit, as per the manufacturer's instructions. The nucleotide mixture comprise the standard nucleotides (A, C, G, U) as well as the modified nucleotides pseudouridine (ty) and 5-methyl-cytidine (5meC, 5 mc or m5C). The transcribed synthetic mRNA is then capped (using e.g., ARCA) and a poly(A) tail is added. Dephosphorylation of 5′-ends of the mRNA is performed by Antarctic Phosphatase and heat-inactivation of the phosphatase. Purification of the synthetic mRNA is performed using MEGAclear transcription clean-up kit from ThermoFisher Scientific™ AM1908. The RNA obtained after in vitro transcription is washed using elution solution, ethanol, and elution buffer according to the manufacturer's protocol. Concentration and purity of eluted mRNA is assessed by evaluating absorbance at 260 and 280 nm. Correct transcript length is verified by running the mRNA on agarose gel electrophoresis under denaturing conditions containing ssRNA ladder as reference. Upon verification, in vitro mRNA is stored at −80° C.

### Example 2

Transfection of MCF7 and MCF7/MDR Cancer Cells with In Vitro Transcribed KEAP1 mRNA

The goal of these experiments is to evaluate the transfectability of various cell lines, as well as evaluate the role of KEAP1 as a tumor cells sensitizer, evaluate its role in decreasing the viability of tumor cells. The synthetic mRNA produced in Example 1, or the cLUC mRNA are transfected in cancer cells using the Lipofectamine MessengerMAX™ Transfection Reagent, ThermoFisher™, Cat No: LMRNA001. 0, 0.3, 1, 3 or 9 ng of either mRNA is used per 1000 cells.

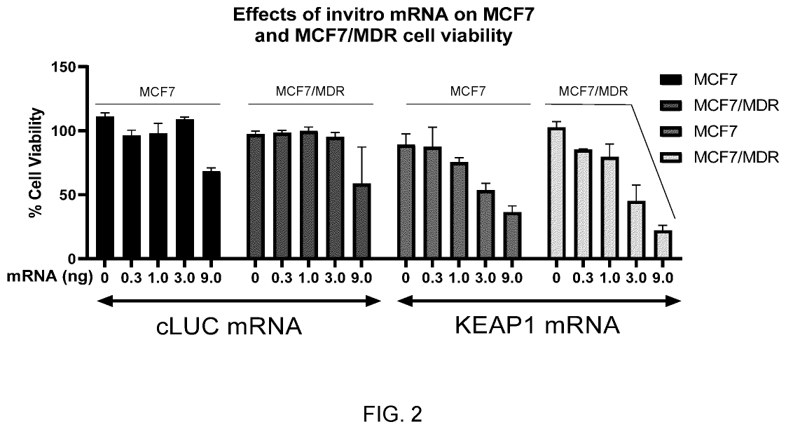

MCF7 and MCF7/MDR human breast adenocarcinoma cell lines are transfected for 48 hours with 0, 0.3, 1, 3 and 9 ng of KEAP1 mRNA, or with 0, 0.3, 1, 3 and 9 ng of a control Cypridina luciferase (cLUC) (SEQ ID NO: 10) mRNA. The cells viability was measured using the CellTiter-Glo® assay from Promega®. KEAP1 mRNA specific effects on MCF7 and MCF7/MDR cells survival were observed when cells were transfected with 0, 0.3, 1, 3 and 9 ng of KEAP1 mRNA. Now referring to FIG. 2, it can be seen that increasing concentrations of the control mRNA cLUC had little effect on cell survival unless a large amount of 9 ng was transfected, while KEAP1 mRNA caused a dose dependent decrease in viability in both MCF7 and MCF7/MDR human breast adenocarcinoma cells.

### Example 3

Transfection of Mcf7 and MCF7/MDR Cancer Cells with In Vitro Transcribed KEAP1 mRNA

The synthetic mRNA produced in Example 1 is transfected in cancer cells using the Lipofectamine MessengerMAX™ Transfection Reagent.

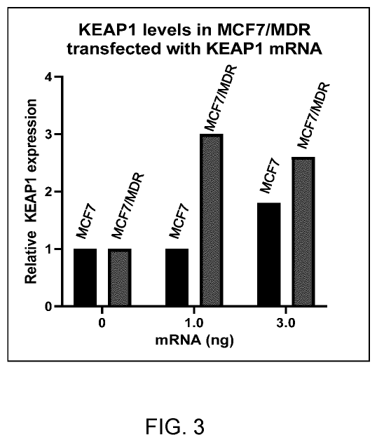

MCF7 and MCF7/MDR human breast adenocarcinoma cell lines are transfected for 48 hours with 0, 1 and 3 ng of KEAP1 mRNA, or with 0, 1 and 3 ng of a control Cypridina luciferase (cLUC) mRNA, per 1000 cells. The cells viability was measured using the CellTiter-Glo® assay from Promega®. In-cell Western analysis is used to detect the presence of KEAP1. Around 2-fold to 3-fold induction of KEAP1 protein was observed in both cell lines transfected with KEAP1 mRNA (FIG. 3).

TABLE 1
MCF7 and MCF7/MDR cancer cell lines
survival and protein expression

| | | % Cell | % Cell | Confirmed |
|---|---|---|---|---|
| | | survival | survival | KEAP1 |
| Cell | mRNA | cLUC | KEAP1 | protein |
| line | (ng) | mRNA | mRNA | expression |
| MCF7 | 0 | 100 | 100 | >3x (3 ng mRNA), |
| | 1 | 100 | 40 | 48 hours |
| | 3 | 55 | 25 | transfection |
| MCF7/MDR | 0 | 100 | 100 | 3x (1 ng and |
| | 1 | 100 | 40 | 3 ng mRNA), |
| | 3 | 55 | 20 | 48 hours |
| | | | | transfection |

Both MCF7 and MCF7/MDR cells were sensitive to KEAP1 mRNA at 1 and 3 ng of mRNA compared to control (cLUC).

### Example 4

Transfection of Human Mammary Epithelial Cells (HMEC) and MCF7 Cancer Cells with In Vitro Transcribed KEAP1 mRNA

The synthetic mRNA produced in Example 1 is transfected in cancer cells using the Lipofectamine MessengerMAX™ Transfection Reagent.

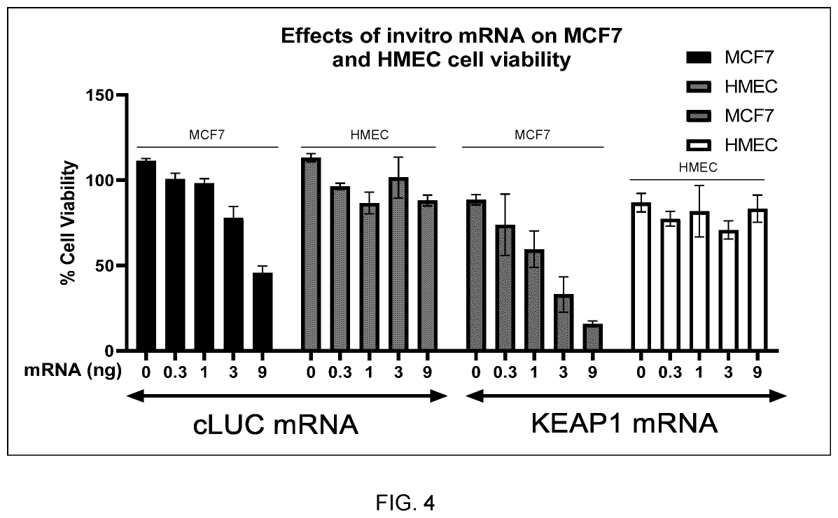

Human Mammary Epithelial Cells (HMEC), which are non-cancer cells were compared to MCF7 cancer cells. The HMEC cells and MCF7 cancer cells were transfected for 48 hours with 0, 0.3, 1, 3 and 9 ng of KEAP1 mRNA, or a control cLUC mRNA, per 1000 cells and their respective survival were compared. The cells viability was measured using the CellTiter-Glo® assay from Promega®. The results shown in Table 2 show that KEAP1 mRNA did not decrease cell viability in HMEC cells. Now referring to FIG. 4, cell viability of MCF7 cells was affected by KEAP1 mRNA, but the viability of the HMEC cells was unaffected by KEAP1 mRNA.

TABLE 2
HMEC and MCF7 cancer cell lines survival and protein expression

| | | % Cell | % Cell | Confirmed |
|---|---|---|---|---|
| | | survival | survival | KEAP1 |
| Cell | mRNA | cLUC | KEAP1 | protein |
| line | (ng) | mRNA | mRNA | expression |
| MCF7 | 0 | 100 | 100 | >3x (3 ng mRNA), |
| | 3 | 100 | 40 | 48 hours |
| | 9 | 55 | 25 | transfection |
| HMEC | 0 | Resistant | Resistant | >3x (3 ng mRNA), |
| | 3 | | | 48 hours |
| | 9 | | | transfection |

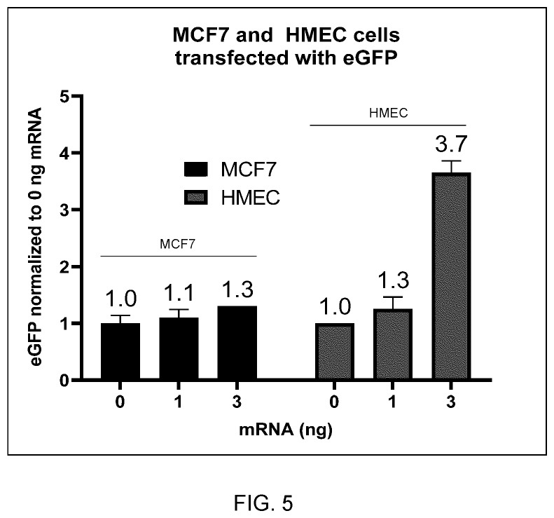

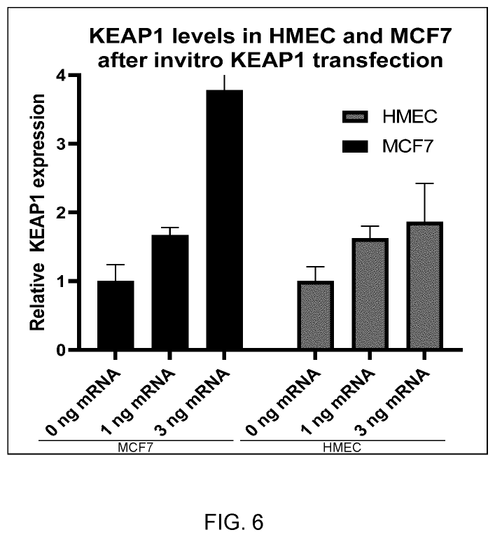

To confirm that this result was not caused by a resistance of the HMEC to transfection, an enhanced GFP (eGFP) (SEQ ID NO: 11) mRNA was transfected under the same conditions. Now referring to FIG. 5. An up to 4× eGFP overexpression was observed in the HMEC cells confirming their transfectability. MCF7 cells also showed similar transfectability. To further confirm these results, the expression of KEAP1 was confirmed by In-cell Western analysis from cells transfected with 0, 1 or 3 ng mRNA. The results showed that around an up to 3-fold induction of KEAP1 protein was observed in HMEC cells lines transfected with KEAP1 mRNA (FIG. 6).

### Example 5

Transfection of OVCAR3 and SUIT2 Cancer Cells with In Vitro Transcribed KEAP1 mRNA

The synthetic mRNA produced in Example 1 is transfected in cancer cells using the Lipofectamine MessengerMAX™ Transfection Reagent.

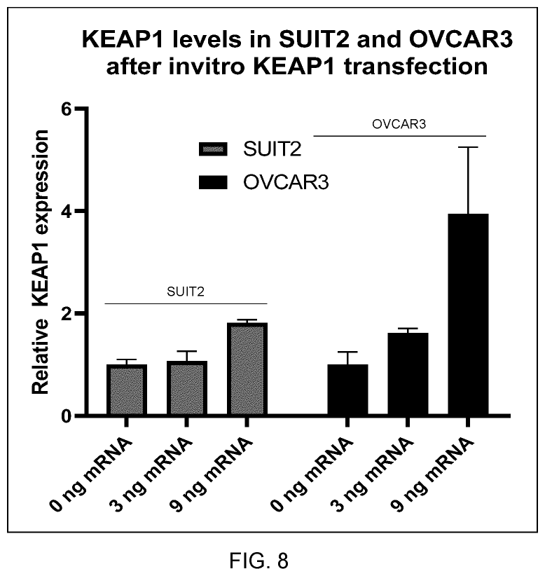

OVCAR3 epithelial cells isolated from malignant ascites of a patient with progressive adenocarcinoma of the ovary, and SUIT2 human pancreatic cancer cell lines are transfected for 24 hours (MCF7 cells) and 48 hours (MCF7/MDR) with 0, 0.3, 1, 3 and 9 ng of KEAP1 mRNA, or with 0, 0.3, 1, 3 and 9 ng of a control Cypridina luciferase (cLUC)(SEQ ID NO: 10) mRNA, per 1000 cells. The cells viability was measured using the CellTiter-Glo® assay from Promega®. In-cell Western analysis is used to detect the presence of KEAP1. Around 2-fold to 4-fold induction of KEAP1 protein was observed in these cells lines transfected with KEAP1 mRNA (FIG. 8).

TABLE 3
OVCAR3and SUIT2 cancer cell lines
survival and protein expression

| | | % Cell | % Cell | Confirmed |
|---|---|---|---|---|
| | | survival | survival | KEAP1 |
| Cell | mRNA | cLUC | KEAP1 | protein |
| line | (ng) | mRNA | mRNA | expression |
| OVCAR3 | 0 | 100 | 100 | 4x (9 ng mRNA), |
| | 3 | 90 | 20 | 24 hours |
| | 9 | 80 | 10 | transfection |
| SUIT2 | 0 | Resistant | 100 | 2x (9 ng mRNA), |
| | 3 | | 100 | 24 hours |
| | 9 | | 50 | transfection |

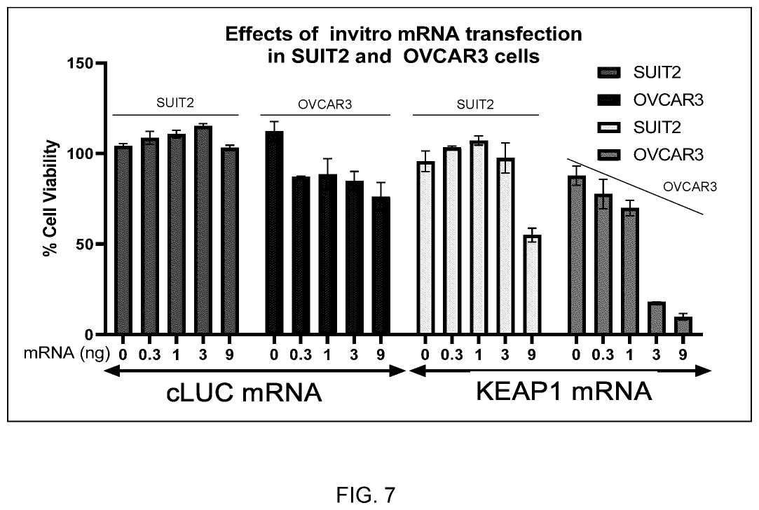

OVCAR3 were sensitive to KEAP1 mRNA at 3 and 9 ng of mRNA compared to control (cLUC). SUIT2 cells were sensitive to KEAP1 mRNA at 9 ng of mRNA compared to control (cLUC). See FIG. 7 and Table 3 above.

While preferred embodiments have been described above and illustrated in the accompanying drawings, it will be evident to those skilled in the art that modifications may be made without departing from this disclosure. Such modifications are considered as possible variants comprised in the scope of the disclosure.

SEQUENCE TABLE

| SEQ ID NO: | Sequence | Description |
|---|---|---|
| SEQ ID NO: 1 | MQPDPRPSGAGACCRFLPLQSQCPEGAGDAVMYASTE | Human KEAP1 |
| | CKAEVTPSQHGNRTFSYTLEDHTKQAFGIMNELRLSQQ | amino acid |
| | LCDVTLQVKYQDAPAAQFMAHKVVLASSSPVFKAMFTN | sequence |
| | GLREQGMEVVSIEGIHPKVMERLIEFAYTASISMGEKCV | |
| | LHVMNGAVMYQIDSVVRACSDFLVQQLDPSNAIGIANFA | |
| | EQIGCVELHQRAREYIYMHFGEVAKQEEFFNLSHCQLV | |
| | TLISRDDLNVRCESEVFHACINWVKYDCEQRRFYVQALL | |
| | RAVRCHSLTPNFLQMQLQKCEILQSDSRCKDYLVKIFEE | |
| | LTLHKPTQVMPCRAPKVGRLIYTAGGYFRQSLSYLEAY | |
| | NPSDGTWLRLADLQVPRSGLAGCVVGGLLYAVGGRNN | |
| | SPDGNTDSSALDCYNPMTNQWSPCAPMSVPRNRIGVG | |
| | VIDGHIYAVGGSHGCIHHNSVERYEPERDEWHLVAPML | |
| | TRRIGVGVAVLNRLLYAVGGFDGTNRLNSAECYYPERN | |
| | EWRMITAMNTIRSGAGVCVLHNCIYAAGGYDGQDQLNS | |
| | VERYDVETETWTFVAPMKHRRSALGITVHQGRIYVLGG | |
| | YDGHTFLDSVECYDPDTDTWSEVTRMTSGRSGVGVAV | |
| | TMEPCRKQIDQQNCTC | |
| SEQ ID NO: 2 | ATGCAGCCAGATCCCAGGCCTAGCGGGGCTGGGGC | Human KEAP1 |
| | CTGCTGCCGATTCCTGCCCCTGCAGTCACAGTGCCC | nucleotide |
| | TGAGGGGGCAGGGGACGCGGTGATGTACGCCTCCA | sequence |
| | CTGAGTGCAAGGCGGAGGTGACGCCCTCCCAGCATG | |
| | GCAACCGCACCTTCAGCTACACCCTGGAGGATCATA | |
| | CCAAGCAGGCCTTTGGCATCATGAACGAGCTGCGGC | |
| | TCAGCCAGCAGCTGTGTGACGTCACACTGCAGGTCA | |
| | AGTACCAGGATGCACCGGCCGCCCAGTTCATGGCCC | |
| | ACAAGGTGGTGCTGGCCTCATCCAGCCCTGTCTTCAA | |
| | GGCCATGTTCACCAACGGGCTGCGGGAGCAGGGCAT | |
| | GGAGGTGGTGTCCATTGAGGGTATCCACCCCAAGGT | |
| | CATGGAGCGCCTCATTGAATTCGCCTACACGGCCTC | |
| | CATCTCCATGGGCGAGAAGTGTGTCCTCCACGTCAT | |
| | GAACGGTGCTGTCATGTACCAGATCGACAGCGTTGT | |
| | CCGTGCCTGCAGTGACTTCCTGGTGCAGCAGCTGGA | |
| | CCCCAGCAATGCCATCGGCATCGCCAACTTCGCTGA | |
| | GCAGATTGGCTGTGTGGAGTTGCACCAGCGTGCCCG | |
| | GGAGTACATCTACATGCATTTTGGGGAGGTGGCCAA | |
| | GCAAGAGGAGTTCTTCAACCTGTCCCACTGCCAACTG | |
| | GTGACCCTCATCAGCCGGGACGACCTGAACGTGCGC | |
| | TGCGAGTCCGAGGTCTTCCACGCCTGCATCAACTGG | |
| | GTCAAGTACGACTGCGAACAGCGACGGTTCTACGTC | |
| | CAGGCGCTGCTGCGGGCCGTGCGCTGCCACTCGTT | |
| | GACGCCGAACTTCCTGCAGATGCAGCTGCAGAAGTG | |
| | CGAGATCCTGCAGTCCGACTCCCGCTGCAAGGACTA | |
| | CCTGGTCAAGATCTTCGAGGAGCTCACCCTGCACAA | |
| | GCCCACGCAGGTGATGCCCTGCCGGGCGCCCAAGG | |
| | TGGGCCGCCTGATCTACACCGCGGGCGGCTACTTCC | |
| | GACAGTCGCTCAGCTACCTGGAGGCTTACAACCCCA | |
| | GTGACGGCACCTGGCTCCGGTTGGCGGACCTGCAG | |
| | GTGCCGCGGAGCGGCCTGGCCGGCTGCGTGGTGGG | |
| | CGGGCTGTTGTACGCCGTGGGGGGCAGGAACAACTC | |
| | GCCCGACGGCAACACCGACTCCAGCGCCCTGGACTG | |
| | TTACAACCCCATGACCAATCAGTGGTCGCCCTGCGC | |
| | CCCCATGAGCGTGCCCCGTAACCGCATCGGGGTGG | |
| | GGGTCATCGATGGCCACATCTATGCCGTCGGCGGCT | |
| | CCCACGGCTGCATCCACCACAACAGTGTGGAGAGGT | |
| | ATGAGCCAGAGCGGGATGAGTGGCACTTGGTGGCCC | |
| | CAATGCTGACACGAAGGATCGGGGTGGGCGTGGCTG | |
| | TCCTCAATCGTCTCCTTTATGCCGTGGGGGGCTTTGA | |
| | CGGGACAAACCGCCTTAATTCAGCTGAGTGTTACTAC | |
| | CCAGAGAGGAACGAGTGGCGAATGATCACAGCAATG | |
| | AACACCATCCGAAGCGGGGCAGGCGTCTGCGTCCTG | |
| | CACAACTGTATCTATGCTGCTGGGGGCTATGATGGTC | |
| | AGGACCAGCTGAACAGCGTGGAGCGCTACGATGTGG | |
| | AAACAGAGACGTGGACTTTCGTAGCCCCCATGAAGC | |
| | ACCGGCGAAGTGCCCTGGGGATCACTGTCCACCAGG | |
| | GGAGAATCTACGTCCTTGGAGGCTATGATGGTCACAC | |
| | GTTCCTGGACAGTGTGGAGTGTTACGACCCAGATACA | |
| | GACACCTGGAGCGAGGTGACCCGAATGACATCGGGC | |
| | CGGAGTGGGGTGGGCGTGGCTGTCACCATGGAGCC | |
| | CTGCCGGAAGCAGATTGACCAGCAGAACTGTACCTG | |
| | T | |
| SEQ ID NO: 3 | EVTPSQHGNRTFSYTLEDHTKQAFGIMNELRLSQQLCD | BTB domain of |
| | VTLQVKYQDAPAAQFMAHKVVLASSSPVFKAMFTNGLR | KEAP1 amino |
| | EQGMEVVSIEGIHPKVMERLIEFAYTASISMGEKCVLH | acid sequence |
| | VMNGAVMYQIDSVVRACSDF | |
| SEQ ID NO: 4 | GAGGTGACGCCCTCCCAGCATGGCAACCGCACCTTC | BTB domain of |
| | AGCTACACCCTGGAGGATCATACCAAGCAGGCCTTT | KEAP1 |
| | GGCATCATGAACGAGCTGCGGCTCAGCCAGCAGCTG | nucleotide |
| | TGTGACGTCACACTGCAGGTCAAGTACCAGGATGCA | sequence |
| | CCGGCCGCCCAGTTCATGGCCCACAAGGTGGTGCTG | |
| | GCCTCATCCAGCCCTGTCTTCAAGGCCATGTTCACC | |
| | AACGGGCTGCGGGAGCAGGGCATGGAGGTGGTGTCC | |
| | ATTGAGGGTATCCACCCCAAGGTCATGGAGCGCCTC | |
| | ATTGAATTCGCCTACACGGCCTCCATCTCCATGGGC | |
| | GAGAAGTGTGTCCTCCACGTCATGAACGGTGCTGTC | |
| | ATGTACCAGATCGACAGCGTTGTCCGTGCCTGCAGT | |
| | GACTTC | |
| SEQ ID NO: 5 | IYTAGGYFRQSLSYLEAYNPSDGTWLRLADLQVPRSGL | Kelch domain of |
| | AGCVVGGLLYAVGGRNNSPDGNTDSSALDCYNPMTNQ | KEAP1 amino |
| | WSPCAPMSVPRNRIGVGVIDGHIYAVGGSHGCIHHNSV | acid sequence |
| | ERYEPERDEWHLVAPMLTRRIGVGVAVLNRLLYAVGGF | |
| | DGTNRLNSAECYYPERNEWRMITAMNTIRSGAGVCVLH | |
| | NCIYAAGGYDGQDQLNSVERYDVETETWTFVAPMKHR | |
| | RSALGITVHQGRIYVLGGYDGHTFLDSVECYDPDTDTW | |
| | SEVTRMTSGRSGVGVAVTMEPCRKQIDQQNCTC | |
| SEQ ID NO: 6 | ATCTACACCGCGGGCGGCTACTTCCGACAGTCGCTC | Kelch domain of |
| | AGCTACCTGGAGGCTTACAACCCCAGTGACGGCACC | KEAP1 |
| | TGGCTCCGGTTGGCGGACCTGCAGGTGCCGCGGAG | nucleotide |
| | CGGCCTGGCCGGCTGCGTGGTGGGCGGGCTGTTGT | sequence |
| | ACGCCGTGGGCGGCAGGAACAACTCGCCCGACGGC | |
| | AACACCGACTCCAGCGCCCTGGACTGTTACAACCCC | |
| | ATGACCAATCAGTGGTCGCCCTGCGCCCCCATGAGC | |
| | GTGCCCCGTAACCGCATCGGGGTGGGGGTCATCGAT | |
| | GGCCACATCTATGCCGTCGGCGGCTCCCACGGCTGC | |
| | ATCCACCACAACAGTGTGGAGAGGTATGAGCCAGAG | |
| | CGGGATGAGTGGCACTTGGTGGCCCCAATGCTGACA | |
| | CGAAGGATCGGGGTGGGCGTGGCTGTCCTCAATCGT | |
| | CTCCTTTATGCCGTGGGGGGCTTTGACGGGACAAAC | |
| | CGCCTTAATTCAGCTGAGTGTTACTACCCAGAGAGGA | |
| | ACGAGTGGCGAATGATCACAGCAATGAACACCATCC | |
| | GAAGCGGGGCAGGCGTCTGCGTCCTGCACAACTGTA | |
| | TCTATGCTGCTGGGGGCTATGATGGTCAGGACCAGC | |
| | TGAACAGCGTGGAGCGCTACGATGTGGAAACAGAGA | |
| | CGTGGACTTTCGTAGCCCCCATGAAGCACCGGCGAA | |
| | GTGCCCTGGGGATCACTGTCCACCAGGGGAGAATCT | |
| | ACGTCCTTGGAGGCTATGATGGTCACACGTTCCTGGA | |
| | CAGTGTGGAGTGTTACGACCCAGATACAGACACCTG | |
| | GAGCGAGGTGACCCGAATGACATCGGGCCGGAGTG | |
| | GGGTGGGCGTGGCTGTCACCATGGAGCCCTGCCGG | |
| | AAGCAGATTGACCAGCAGAACTGTACCTGT | |
| SEQ ID NO: 7 | GGGSGGGSGGGSGGGSGGGSGG | 22 aa Gly/Ser |
| | | linker |
| SEQ ID NO: 8 | TTTTGTAATACGACTCACTATAGGGCGGCCGGGAATT | 5′ UTR region |
| | CGTCGACTGGATCCGGTACCGAGGAGATCTGCCGCC | sequence 1 |
| | GCGATCGCC | |
| SEQ ID NO: 9 | TTTTGTAATACGACTCACTATAGGGCGGCCGGGAATT | 5′ UTR region |
| | CGTCGACTGGATCCGGTACCGAGGAGATCTGCCGCC | sequence 2 |
| | GCGATCGCC | |
| SEQ ID NO: 10 | GACACTATCCCGGTGATCCATACAAACCGAATCCACC | cDNA sequence |
| | ATGAAGACGTTAATCCTTGCCGTTGCATTGGTCTACT | of Cypridina |
| | GCGTCACTGTTAACTGTCAGGAATGTCCCTACGTAGC | noctiluca |
| | TGACCCTCCAAACACAGTTCCAACTTCCTGTGAAGCT | luciferase |
| | AAAGAAGGAGAATGTATTGATTCCAGCTGTTCCACCT | |
| | GCACCAGAGACATACTATCAGATGGACTGTGTGAAAA | |
| | TAAACCAGGAAAGACATGCTGTAGAATGTGTCAATAC | |
| | GTAATTGAATGCAGAGTAGAAGCTGCGGGATGGTTTA | |
| | GAACATTCTATGGAAAGAGATTCCAGTTCCAGGAACC | |
| | AGGTACATACGTGTTGGGTCAAGGAACCAAGGGAGG | |
| | GGACTGGAAAGTGTCCATTACACTGGAGAACCTGGAT | |
| | GGAACCAAAGGAGCTGTGCTGACCAAGACAAGACTG | |
| | GAAGTGGCTGGAGACATCATTGACATCGCTCAAGCAA | |
| | CTGAGAACCCTATCACTGTAAACGGTGGAGCCGACC | |
| | CCATCATCGCCAACCCGTACACCATCGGTGAGGTCA | |
| | CCATTGCTGTTGTTGAGTTGCCAGGCTACAATATCAC | |
| | CGTCATTGAATTCTTCAAACTGATCGTGATTGACATCC | |
| | TCGGAGGAAGATCTGTAAGGATCGCCCCAGACACAG | |
| | CAAACAAAGGGATGATCTCTGGTCTGTGTGGAGATCT | |
| | AAAAATGATGGAGGATACCGACTTCTCTTCAGATCCC | |
| | GAGCAGCTCGCTATTCAGCCCAAAATCAACCAAGAAT | |
| | TCGACGGTTGTCCACTCTATGGAAATCCTGAAGATAT | |
| | CACATACTGCAAAGGTCTTCTTGAGCCATACAAGGAT | |
| | AGCTGCCGCAACAACATCAACTTCTACTACTACACTC | |
| | TGTCCTGCGCCTTCGCCCGCTGTATGGGTGGAGATG | |
| | AGAGAGCCTCACATGTTGTACTGGACTACAGGGAGA | |
| | CGTGCGCTGCCCCCGAAACCAGAGGTACCTGTGTTT | |
| | TGTCTGGACACACATTCTACGATACATTTGACAAAGC | |
| | AAGATATCAATTCCAGGGTCCCTGCAAGGAGATTCTC | |
| | ATGGCTGCCGACTGTTATTGGAACACTTGGGATGTGA | |
| | AGGTTTCACACAGAAATGTTGACTCTTACACAGAAGT | |
| | AGAGAAAGTACGAATCAGGAAACAATCCACTGTCGTA | |
| | GAACTCATTGTGGATGGAAAACAGATCCTGGTTGGAG | |
| | GAGAAGCCGTGTCCATCCCTTACAGCTCTCAGAACAC | |
| | TTCCATCTACTGGCAAGATGGCGACATCCTGACTACA | |
| | GCCATCCTACCTGAAGCTCTGGTGGTCAAGTTCAACT | |
| | TCAAACAGCTGCTCGTAGTCCATATCAGAGATCCATT | |
| | TGATGGCAAGACCTGCGGTATTTGTGGTAACTACAAC | |
| | CAGGATTTCAGTGATGATTCGTTTGATGCTGAAGGAG | |
| | CCTGTGATCTGACCCCCAACCCACCAGGATGTACCG | |
| | AAGAGCAGAGACCTGAGGCTGAACGACTCTGTAATA | |
| | GCCTATTTGTCGGTCAGAGTGATCTCGATCAAAAATG | |
| | CAACGTATGCTACAAGCCTGACCGTGTTGAACGGTGT | |
| | ATGTACGAGTATTGCCTGAGGGGACAACAGGGATTCT | |
| | GTGACCACGCATGGGAGTTCAAGAAAGAATGCTACAT | |
| | CAAGCATGGAGACACTCTAGAAGTACCAGATGAATGC | |
| | AAATAAAAAGTACAAAGGAATCAAGGAAATAACGGAA | |
| | GAAAAAGAAGGGAAATTATCTGTGTATGTCTATATAA | |
| | CGTGTCTAGTTATGCATTCATTGAACCAAGAGA | |
| SEQ ID NO: 11 | AAGCTTTATTAAAATGTCTAAAGGTGAAGAATTATTCA | cDNA sequence |
| | CTGGTGTTGTCCCAATTTTGGTTGAATTAGATGGTGA | of enhanced GFP |
| | TGTTAATGGTCACAAATTTTCTGTCTCCGGTGAAGGT | |
| | GAAGGTGATGCTACTTACGGTAAATTGACCTTAAAATT | |
| | TATTTGTACTACTGGTAAATTGCCAGTTCCATGGCCAA | |
| | CCTTAGTCACTACTTTCGGTTATGGTGTTCAATGTTTT | |
| | GCTAGATACCCAGATCATATGAAACAACATGACTTTTT | |
| | CAAGTCTGCCATGCCAGAAGGTTATGTTCAAGAAAGA | |
| | ACTATTTTTTTCAAAGATGACGGTAACTACAAGACCAG | |
| | AGCTGAAGTCAAGTTTGAAGGTGATACCTTAGTTAAT | |
| | AGAATCGAATTAAAAGGTATTGATTTTAAAGAAGATGG | |
| | TAACATTTTAGGTCACAAATTGGAATACAACTATAACT | |
| | CTCACAATGTTTACATCATGGCTGACAAACAAAAGAAT | |
| | GGTATCAAAGTTAACTTCAAAATTAGACACAACATTGA | |
| | AGATGGTTCTGTTCAATTAGCTGACCATTATCAACAAA | |
| | ATACTCCAATTGGTGATGGTCCAGTCTTGTTACCAGA | |
| | CAACCATTACTTATCCACTCAATCTGCCTTATCCAAAG | |
| | ATCCAAACGAAAAGAGAGACCACATGGTCTTGTTAGA | |
| | ATTTGTTACTGCTGCTGGTATTACCCATGGTATGGAT | |
| | GAATTGTACAAATAACTGCAG | |

1. A pharmaceutical composition comprising:
a plurality of lipid nanoparticles comprising a cationic lipid, a neutral lipid, a cholesterol, and a PEG lipid, wherein the plurality of lipid nanoparticles has a mean particle size between 80 nm and 160 nm; and wherein the plurality of lipid nanoparticles comprises within their core a synthetic messenger ribonucleic acid (mRNA) comprising:
a) a 5′-cap structure;
b) a 5′-untranslated region (UTR);
c) an open reading frame encoding a Kelch-like ECH-associated protein 1 (KEAP1) protein or a fragment thereof, consisting of nucleotides comprising uracil, cytosine, adenine, guanine, a modified uracil, a modified cytosine, a modified adenine, a modified guanine, or combinations thereof;
d) a 3′-UTR; and
e) a poly(A) region of at least 100 nucleotides in length,
wherein the KEAP1 protein or a fragment thereof is operable to bind to and inhibit release of Nuclear factor erythroid 2-related factor 2 (Nrf2), and presents the Nrf2 for ubiquitination and subsequent degradation.

2. The pharmaceutical composition of claim 1, wherein the cationic lipid is a biodegradable cationic lipid.

3. The pharmaceutical composition of claim 2, wherein the biodegradable cationic lipid comprises an ester linkage.

4. The pharmaceutical composition of claim 3, wherein the biodegradable cationic lipid comprises DLin-DMA with an internal ester, DLin-DMA with a terminal ester, DLin-MC3-DMA with an internal ester, or DLin-MC3-DMA with a terminal ester.

5. The pharmaceutical composition of claim 1, wherein the at least one 5′-cap structure comprises cap1, ARCA, inosine, N1-methyl-guanosine, 2′-fluoro-guanosine, 7-deaza-guanosine, 8-oxo-guanosine, 2-amino-guanosine, LNA-guanosine, 2-azido-guanosine, or a combination thereof.

6. The pharmaceutical composition of claim 5, wherein the at least one 5′-cap structure is cap0, cap1, or ARCA.

7. The pharmaceutical composition of claim 1, wherein the 3′-UTR is an alpha-globin 3′-UTR.

8. The pharmaceutical composition of claim 1, wherein the 5′-UTR comprises a Kozak sequence.

9. The pharmaceutical composition of claim 1, wherein the plurality of lipid nanoparticles has a mean polydispersity index (PDI) of between 0.02 and 0.2.

10. The pharmaceutical composition of claim 1, wherein the plurality of lipid nanoparticles has a mean lipid to polynucleotide, ratio (wt/wt) of between 10 and 20.

11. The pharmaceutical composition claim 1, wherein the modified uracil is N1-methyl-pseudouridine, pseudouridine, or a combination thereof.

12. The pharmaceutical composition of claim 1, wherein the modified uracil is 5-methoxy-uracil.

13. The pharmaceutical composition of claim 1, wherein the modified cytidine is 5-methyl-cytidine.

14. The pharmaceutical composition of claim 1, wherein the open reading frame encoding KEAP1 protein or a fragment thereof comprises SEQ ID NO: 1.

15. The pharmaceutical composition of claim 1, wherein the open reading frame encoding KEAP1 protein comprises the nucleotide sequence of SEQ ID NO: 2.

16. The pharmaceutical composition of claim 1, wherein the open reading frame encoding KEAP1 protein or a fragment thereof is a fragment which comprises a BTB domain of KEAP1 comprising SEQ ID NO: 3 fused to a Kelch domain of KEAP1 comprising SEQ ID NO: 5, and is operable to bind to and inhibit release of Nuclear factor erythroid 2-related factor 2 (Nrf2), and presents the Nrf2 for ubiquitination and subsequent degradation.

17. The pharmaceutical composition of claim 16, wherein the BTB domain of KEAP1 is functionally linked to the Kelch domain of KEAP1 via a peptide linker.

18. The pharmaceutical composition of claim 17, wherein the peptide linker comprises about 3 to about 40 amino acid residues.

19. The compound of claim 17, wherein the peptide linker comprises the amino acid sequence (GGGGS)ₙ or (GGGS)ₙ, wherein n≥1.

20. A method for the treatment of a disease associated with KEAP1 and/or Nrf2 comprising:
(a) systematically administering to a patient in need thereof a therapeutically effective amount of a first therapeutic agent comprising the pharmaceutical composition of claim 1; and
(b) administering to the patient in need thereof a therapeutically effective amount of a second therapeutic agent.

21.-31. (canceled)
